SEQUENCE LISTING (34 sequences)

SEQ ID NO: 1 - protein, 133 aa, Homo sapiens [sequence omitted]

SEQ ID NO: 2 - protein, 132 aa, synthetic construct
  REGION 1..132: note = Synthetic: human IL-2 mutant F42A
APTSSSTKKTQLQLEHLLLDLQMILNGINNYKNPKLTRMLTAKFYMPKKATEKHLQCLEEELKPLEEVLNLAQSKNFHLRPRDLISNINVIVLELKGSETTFMCEYADETATIVEFLNRWITFCQSIISTLT

SEQ ID NO: 3 - protein, 133 aa, synthetic construct
  REGION 1..133: note = Synthetic: human IL-2 mutant F42AV1
APTSSSTKKTQLQLEHLLLDLQMILNGINNYKNPKLTRMLTAKFYMPKKATELKHLQCLEEELKPLEEVLNLAQSKNFHLEPRDIISNINVIVLELKGSETTFMCEYADETATIVEFLNRWITFCQSIISTLT

SEQ ID NO: 4 - protein, 133 aa, synthetic construct
  REGION 1..133: note = Synthetic: human IL-2 mutant F42AV2
APTSSSTKKTQLQLEHLLLDLQMILNGINNYKNPKLTRMLTAKFYMPKKATELKHLQCLEEELKPLEEVLNLAQSKNFHLEPRDTISNINVIVLELKGSETTFMCEYADETATIVEFLNRWITFCQSIISTLT

SEQ ID NO: 5 - protein, 227 aa, Homo sapiens
  REGION 1..227: note = misc_feature - human IgG1 Fc, human Fc, dimeric, wild-type
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 227 aa, synthetic construct
  REGION 1..227: note = Synthetic: human IgG1 Fc (human Fc, dimeric, mutant)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 217 aa, Homo sapiens
  REGION 1..217: note = misc_feature - human IgG1 Fc (human Fc, monomeric, wild-type)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 8 - protein, 217 aa, synthetic construct
  REGION 1..217: note = Synthetic: human IgG1 Fc (human Fc, monomeric, mutant)
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 9 - protein, 227 aa, synthetic construct
  REGION 1..227: note = Synthetic: human IgG1 Fc (human Fc6, mutant)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 10 - protein, 227 aa, synthetic construct
  REGION 1..227: note = Synthetic: human IgG1 Fc (human Fc9, mutant)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 11 - protein, 449 aa, synthetic construct
  REGION 1..449: note = Synthetic: anti-Claudin antibody heavy chain
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCARVYYGNSFAYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 220 aa, synthetic construct
  REGION 1..220: note = Synthetic: anti-Claudin antibody light chain
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYFYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 587 aa, synthetic construct
  REGION 1..587: note = Synthetic: anti-Claudin antibody heavy chain-IL-2F42AV1
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCARVYYGNSFAYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSAPTSSSTKKTQLQLEHLLLDLQMILNGINNYKNPKLTRMLTAKFYMPKKATELKHLQCLEEELKPLEEVLNLAQSKNFHLEPRDIISNINVIVLELKGSETTFMCEYADETATIVEFLNRWITFCQSIISTLT

SEQ ID NO: 14 - protein, 586 aa, synthetic construct
  REGION 1..586: note = Synthetic: anti-Claudin antibody heavy chain-IL-2F42AV2
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCARVYYGNSFAYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSAPTSSSTKKTQLQLEHLLLDLQMILNGINNYKNPKLTRMLTAKFYMPKKATELKHLQCLEEELKPLEEVLNLAQSKNFHLEPRDTISNINVIVLELKGSETTFMCEYADETATIVEFLNRWITFCQSIISTLT

SEQ ID NO: 15 - protein, 364 aa, synthetic construct
  REGION 1..364: note = Synthetic: human IL-2 mutant F42AV2-human IgG1 Fc (human Fc, dimeric, wild-type)
APTSSSTKKTQLQLEHLLLDLQMILNGINNYKNPKLTRMLTAKFYMPKKATELKHLQCLEEELKPLEEVLNLAQSKNFHLEPRDTISNINVIVLELKGSETTFMCEYADETATIVEFLNRWITFCQSIISTLTGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 354 aa, synthetic construct
  REGION 1..354: note = Synthetic: human IgG1 Fc (human Fc, monomeric, wild-type)- human IL-2 mutant F42AV2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGKGGGSAPTSSSTKKTQLQLEHLLLDLQMILNGINNYKNPKLTRMLTAKFYMPKKATELKHLQCLEEELKPLEEVLNLAQSKNFHLEPRDTISNINVIVLELKGSETTFMCEYADETATIVEFLNRWITFCQSIISTLT

SEQ ID NO: 17 - protein, 589 aa, synthetic construct
  REGION 1..589: note = Synthetic: human IL-2 mutant F42AV2-human IgG1 Fc (human Fc, dimeric, wild-type)- anti-Claudin antibody heavy chain
APTSSSTKKTQLQLEHLLLDLQMILNGINNYKNPKLTRMLTAKFYMPKKATELKHLQCLEEELKPLEEVLNLAQSKNFHLEPRDTISNINVIVLELKGSETTFMCEYADETATIVEFLNRWITFCQSIISTLTGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCARVYYGNSFAYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 18 - DNA, 399 bp, Homo sapiens
  misc_feature 1..399: note = human IL-2WT nucleic acid sequence
  source 1..399: mol_type = genomic DNA
gcccctacaagcagcagcaccaagaagacccagctgcagctggaacacctgctgctggatctgcagatgatcctgaacggcatcaacaactacaagaaccccaagctgacccggatgctgaccttcaagttctacatgcccaagaaggccaccgagctgaagcacctccagtgtctggaggaggagctgaagcctctggaggaagtgctgaacctggcccagagcaagaacttccacttaagacccagggacttaatctccaacatcaacgtgatagtgctggaactgaagggcagcgagaccaccttcatgtgcgagtacgccgacgagaccgctaccatcgtggagttcctgaaccgctggatcaccttttgccagagcatcatcagcacactgacc

SEQ ID NO: 19 - DNA, 399 bp, synthetic construct
  misc_feature 1..399: note = Synthetic: human IL-2 mutant F42A
  source 1..399: mol_type = other DNA
gcccctacaagcagcagcaccaagaagacccagctgcagctggaacacctgctgctggatctgcagatgatcctgaacggcatcaacaactacaagaaccccaagctgacccggatgctgaccgccaagttctacatgcccaagaaggccaccgagctgaagcacctccagtgtctggaggaggagctgaagcctctggaggaagtgctgaacctggcccagagcaagaacttccacttaagacccagggacttaatctccaacatcaacgtgatagtgctggaactgaagggcagcgagaccaccttcatgtgcgagtacgccgacgagaccgctaccatcgtggagttcctgaaccgctggatcaccttttgccagagcatcatcagcacactgacc

SEQ ID NO: 20 - DNA, 399 bp, synthetic construct
  misc_feature 1..399: note = Synthetic: human IL-2 mutant F42AV1
  source 1..399: mol_type = other DNA
gcccctacaagcagcagcaccaagaagacccagctgcagctggaacacctgctgctggatctgcagatgatcctgaacggcatcaacaactacaagaaccccaagctgacccggatgctgaccgccaagttctacatgcccaagaaggccaccgagctgaagcacctccagtgtctggaggaggagctgaagcctctggaggaagtgctgaacctggcccagagcaagaacttccacttagaacccagggacataatctccaacatcaacgtgatagtgctggaactgaagggcagcgagaccaccttcatgtgcgagtacgccgacgagaccgctaccatcgtggagttcctgaaccgctggatcaccttttgccagagcatcatcagcacactgacc

SEQ ID NO: 21 - DNA, 399 bp, synthetic construct
  misc_feature 1..399: note = Synthetic: human IL-2 mutant F42AV2
  source 1..399: mol_type = other DNA
gcccctacaagcagcagcaccaagaagacccagctgcagctggaacacctgctgctggatctgcagatgatcctgaacggcatcaacaactacaagaaccccaagctgacccggatgctgaccgccaagttctacatgcccaagaaggccaccgagctgaagcacctccagtgtctggaggaggagctgaagcctctggaggaagtgctgaacctggcccagagcaagaacttccacttagaacccagggacacaatctccaacatcaacgtgatagtgctggaactgaagggcagcgagaccaccttcatgtgcgagtacgccgacgagaccgctaccatcgtggagttcctgaaccgctggatcaccttttgccagagcatcatcagcacactgacc

SEQ ID NO: 22 - DNA, 681 bp, Homo sapiens
  misc_feature 1..681: note = human IgG1 Fc human Fc, dimeric, wild-type
  source 1..681: mol_type = genomic DNA
gacaagacccacacttgccccccttgtcccgctccggaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 23 - DNA, 681 bp, synthetic construct
  misc_feature 1..681: note = Synthetic: human IgG1 Fc (human Fc, dimeric, mutant)
  source 1..681: mol_type = other DNA
gacaaaactcacacatgcccaccgtgcccagcaccggaagccgcaggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 24 - DNA, 651 bp, Homo sapiens
  misc_feature 1..651: note = human IgG1 Fc (human Fc, monomeric, wild-type)
  source 1..651: mol_type = genomic DNA
gcgccggagctgctcggtggaccatcagtattcctcttcccaccgaagcctaaggatactctcatgatatccaggactcctgaggttacttgtgttgtggttgacgtatctcatgaggaccctgaggtcaaatttaattggtatgtagacggagtggaagtccacaatgccaagactaaaccaagggaggagcagtataactctacctaccgcgttgtgtcagtgctgacggtgttgcatcaagactggctgaacgggaaggaatacaaatgtaaggtgtccaacaaggccctccctgcgcctatagaaaaaactataagtaaagccaaaggtcaaccaagagaacctcaggtatatacgaaaccaccctccagggacgagcttaccaagaaccaggtttcactgagttgtctcgtcaaggggttctacccaagcgatattgcggtcgagtgggagtctaatggacaaccggagaataactacaaaactaccgtacctgtgctggatagcgatgggtctttcagactcgcctcttacttgacggtcgataaaagtcgatggcagcagggaaacgtctttagctgcagtgtcatgcatgaagcactccacaaccattacactcagaaaagtctttctctgagtccaggtaag

SEQ ID NO: 25 - DNA, 651 bp, synthetic construct
  misc_feature 1..651: note = Synthetic: human IgG1 Fc (human Fc, monomeric, mutant)
  source 1..651: mol_type = other DNA
gccccggaagcagctggcggcccatccgtattcctttttcctcccaaacccaaggacacgctgatgattagccgaacaccggaggtgacgtgcgttgttgtggatgtgagccacgaagacccagaggtcaagtttaattggtacgtagacggcgttgaggtacataatgccaagacgaaacctagagaggagcagtataactctacatatcgagtggtttcagtgcttactgttttgcatcaggattggttgaatggtaaggaatacaagtgtaaagtgtcaaacaaagcactgggggcaccgatagaaaaaactatcagtaaggcaaaaggacaaccgcgagaaccacaagtatatacaaaacccccttcccgagacgaacttactaagaaccaagtaagtctgtcctgtttggtaaaaggcttttacccctcagacatagccgtagagtgggaatctaacggccaacctgagaataattacaagactacagttccggtactggattctgatggcagttttaggttggcgtcctatttgaccgttgataagtcccggtggcagcaaggaaatgttttttcctgtagtgtgatgcatgaagctcttcacaatcattatacgcaaaagtctttgtccttgtcccctgggaag

SEQ ID NO: 26 - DNA, 681 bp, synthetic construct
  misc_feature 1..681: note = Synthetic: human IgG1 Fc (human Fc6, mutant)
  source 1..681: mol_type = other DNA
gacaagacccacacttgccccccttgtcccgctccggaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgtaccctgcccccatcccgggatgagctgaccaagaaccaggtcagcctgagttgcgcggtcaaaggcttctatcccagcgacatcgccgtggagtgggagagcaatgggcagccggagaacaactacaagaccacgcctcccgtgttggactccgacggctccttcaagctcgtcagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 27 - DNA, 681 bp, synthetic construct
  misc_feature 1..681: note = Synthetic: human IgG1 Fc (human Fc9, mutant)
  source 1..681: mol_type = other DNA
gacaaaacccacacatgcccaccttgtcccgcccctgaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgtcgggatgagctgaccaagaaccaggtcagcctgtggtgcctggtcaaaggcttctatcccagcgacatcgccgtggagtgggagagcaatgggcagccggagaacaactacaagaccacgcctcccgtgttggactccgacggctccttcttcctctacagcgcgctcaccgtggacaagagcaggtggcagcaggggaacgtcttctcatgctccgtgatgcatgaggctctgcacaaccactacacgcagaagagcctctccctgtctccgggtaaa

SEQ ID NO: 28 - DNA, 1344 bp, synthetic construct
  misc_feature 1..1344: note = Synthetic: anti-Claudin antibody heavy chain
  source 1..1344: mol_type = other DNA
caagtgcagctgcagcagcccggcgccgagctggtgagacccggcgcctccgtgaagctgagctgcaaagctagcggctacaccttcacctcctactggatcaactgggtgaagcagagacccggccaaggactggagtggatcggcaacatctacccctccgactcctacaccaactacaaccagaagtttaaggacaaggccacactgaccgtggacaagtcctccagcaccgcctacatgcagctgtcctcccctacctccgaggacagcgccgtgtactactgcgctagagtctactacggcaacagcttcgcctactggggccaaggcaccacactgaccgtgagcagcgccagcactaaggggccctctgtgtttccactcgccccttctagcaaaagcacttccggaggaactgccgctctgggctgtctggtgaaagattacttccccgaaccagtcactgtgtcatggaactctggagcactgacatctggagttcacacctttcctgctgtgctgcagagttctggactgtactccctgtcatctgtggtcaccgtgccatcttcatctctggggacccagacctacatctgtaacgtgaaccacaaaccctccaacacaaaagtggacaaacgagtcgaaccaaaatcttgtgacaaaacccacacatgcccaccttgtcccgcccctgaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 29 - DNA, 660 bp, synthetic construct
  misc_feature 1..660: note = Synthetic: anti-Claudin antibody light chain
  source 1..660: mol_type = other DNA
gacatcgtgatgacccagtcccctagctctctgaccgtgacagccggagagaaagtgaccatgagctgcaagagcagccaatctctgctgaacagcggcaaccagaagaactatctgacatggtatcagcagaagcccggccaaccccccaagctgctgatttactgggccagcacaagagagagcggagtgcccgataggttcaccggcagcggcagcggcaccgatttcacactgaccatctccagcgtgcaagccgaagacctcgccgtctactactgccagaacgactacttctaccctttcaccttcggctccggcaccaaactggagatcaagcgtacggtggctgcaccatctgtcttcatcttcccgccatctgatgagcagttgaaatctggtaccgctagcgttgtgtgcctgctgaataacttttatccacgggaggctaaggtgcagtggaaagtggacaatgccctccagagcggaaatagccaagagtccgttaccgaacaggactctaaagactctacatactccctgtcctccacactgaccctctccaaggccgactatgagaaacacaaggtttacgcatgcgaggtcacacaccagggactctcctctcccgtgaccaagagcttcaaccggggagaatgc

SEQ ID NO: 30 - DNA, 1758 bp, synthetic construct
  misc_feature 1..1758: note = Synthetic: anti-Claudin antibody heavy chain-IL-2F42AV1
  source 1..1758: mol_type = other DNA
caagtgcagctgcagcagcccggcgccgagctggtgagacccggcgcctccgtgaagctgagctgcaaagctagcggctacaccttcacctcctactggatcaactgggtgaagcagagacccggccaaggactggagtggatcggcaacatctacccctccgactcctacaccaactacaaccagaagtttaaggacaaggccacactgaccgtggacaagtcctccagcaccgcctacatgcagctgtcctcccctacctccgaggacagcgccgtgtactactgcgctagagtctactacggcaacagcttcgcctactggggccaaggcaccacactgaccgtgagcagcgccagcactaaggggccctctgtgtttccactcgccccttctagcaaaagcacttccggaggaactgccgctctgggctgtctggtgaaagattacttccccgaaccagtcactgtgtcatggaactctggagcactgacatctggagttcacacctttcctgctgtgctgcagagttctggactgtactccctgtcatctgtggtcaccgtgccatcttcatctctggggacccagacctacatctgtaacgtgaaccacaaaccctccaacacaaaagtggacaaacgagtcgaaccaaaatcttgtgacaaaacccacacatgcccaccttgtcccgcccctgaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggaggaggtagcgcccctacaagcagcagcaccaagaagacccagctgcagctggaacacctgctgctggatctgcagatgatcctgaacggcatcaacaactacaagaaccccaagctgacccggatgctgaccgccaagttctacatgcccaagaaggccaccgagctgaagcacctccagtgtctggaggaggagctgaagcctctggaggaagtgctgaacctggcccagagcaagaacttccacttagaacccagggacataatctccaacatcaacgtgatagtgctggaactgaagggcagcgagaccaccttcatgtgcgagtacgccgacgagaccgctaccatcgtggagttcctgaaccgctggatcaccttttgccagagcatcatcagcacactgacc

SEQ ID NO: 31 - DNA, 1758 bp, synthetic construct
  misc_feature 1..1758: note = Synthetic: anti-Claudin antibody heavy chain-IL-2F42AV2
  source 1..1758: mol_type = other DNA
caagtgcagctgcagcagcccggcgccgagctggtgagacccggcgcctccgtgaagctgagctgcaaagctagcggctacaccttcacctcctactggatcaactgggtgaagcagagacccggccaaggactggagtggatcggcaacatctacccctccgactcctacaccaactacaaccagaagtttaaggacaaggccacactgaccgtggacaagtcctccagcaccgcctacatgcagctgtcctcccctacctccgaggacagcgccgtgtactactgcgctagagtctactacggcaacagcttcgcctactggggccaaggcaccacactgaccgtgagcagcgccagcactaaggggccctctgtgtttccactcgccccttctagcaaaagcacttccggaggaactgccgctctgggctgtctggtgaaagattacttccccgaaccagtcactgtgtcatggaactctggagcactgacatctggagttcacacctttcctgctgtgctgcagagttctggactgtactccctgtcatctgtggtcaccgtgccatcttcatctctggggacccagacctacatctgtaacgtgaaccacaaaccctccaacacaaaagtggacaaacgagtcgaaccaaaatcttgtgacaaaacccacacatgcccaccttgtcccgcccctgaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggaggaggtagcgcccctacaagcagcagcaccaagaagacccagctgcagctggaacacctgctgctggatctgcagatgatcctgaacggcatcaacaactacaagaaccccaagctgacccggatgctgaccgccaagttctacatgcccaagaaggccaccgagctgaagcacctccagtgtctggaggaggagctgaagcctctggaggaagtgctgaacctggcccagagcaagaacttccacttagaacccagggacacaatctccaacatcaacgtgatagtgctggaactgaagggcagcgagaccaccttcatgtgcgagtacgccgacgagaccgctaccatcgtggagttcctgaaccgctggatcaccttttgccagagcatcatcagcacactgacc

SEQ ID NO: 32 - DNA, 1095 bp, synthetic construct
  misc_feature 1..1095: note = Synthetic: human IL-2 mutant F42AV2-human IgG1 Fc (human Fc, dimeric, wild-type)
  source 1..1095: mol_type = other DNA
gcccctacaagcagcagcaccaagaagacccagctgcagctggaacacctgctgctggatctgcagatgatcctgaacggcatcaacaactacaagaaccccaagctgacccggatgctgaccgccaagttctacatgcccaagaaggccaccgagctgaagcacctccagtgtctggaggaggagctgaagcctctggaggaagtgctgaacctggcccagagcaagaacttccacttagaacccagggacacaatctccaacatcaacgtgatagtgctggaactgaagggcagcgagaccaccttcatgtgcgagtacgccgacgagaccgctaccatcgtggagttcctgaaccgctggatcaccttttgccagagcatcatcagcacactgaccggtggaggaggtagcgacaagacccacacttgccccccttgtcccgctccggaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 33 - DNA, 1065 bp, synthetic construct
  misc_feature 1..1065: note = Synthetic: human IgG1 Fc (human Fc, monomeric, wild-type)- human IL-2 mutant F42AV2
  source 1..1065: mol_type = other DNA
gcgccggagctgctcggtggaccatcagtattcctcttcccaccgaagcctaaggatactctcatgatatccaggactcctgaggttacttgtgttgtggttgacgtatctcatgaggaccctgaggtcaaatttaattggtatgtagacggagtggaagtccacaatgccaagactaaaccaagggaggagcagtataactctacctaccgcgttgtgtcagtgctgacggtgttgcatcaagactggctgaacgggaaggaatacaaatgtaaggtgtccaacaaggccctccctgcgcctatagaaaaaactataagtaaagccaaaggtcaaccaagagaacctcaggtatatacgaaaccaccctccagggacgagcttaccaagaaccaggtttcactgagttgtctcgtcaaggggttctacccaagcgatattgcggtcgagtgggagtctaatggacaaccggagaataactacaaaactaccgtacctgtgctggatagcgatgggtctttcagactcgcctcttacttgacggtcgataaaagtcgatggcagcagggaaacgtctttagctgcagtgtcatgcatgaagcactccacaaccattacactcagaaaagtctttctctgagtccaggtaagggtggaggaggtagcgcccctacaagcagcagcaccaagaagacccagctgcagctggaacacctgctgctggatctgcagatgatcctgaacggcatcaacaactacaagaaccccaagctgacccggatgctgaccgccaagttctacatgcccaagaaggccaccgagctgaagcacctccagtgtctggaggaggagctgaagcctctggaggaagtgctgaacctggcccagagcaagaacttccacttagaacccagggacacaatctccaacatcaacgtgatagtgctggaactgaagggcagcgagaccaccttcatgtgcgagtacgccgacgagaccgctaccatcgtggagttcctgaaccgctggatcaccttttgccagagcatcatcagcacactgacc

SEQ ID NO: 34 - DNA, 1773 bp, synthetic construct
  misc_feature 1..1773: note = Synthetic: human IL-2 mutant F42AV2-human IgG1 Fc (human Fc, dimeric, wild-type)- anti-Claudin antibody heavy chain
  source 1..1773: mol_type = other DNA
gcccctacaagcagcagcaccaagaagacccagctgcagctggaacacctgctgctggatctgcagatgatcctgaacggcatcaacaactacaagaaccccaagctgacccggatgctgaccgccaagttctacatgcccaagaaggccaccgagctgaagcacctccagtgtctggaggaggagctgaagcctctggaggaagtgctgaacctggcccagagcaagaacttccacttagaacccagggacacaatctccaacatcaacgtgatagtgctggaactgaagggcagcgagaccaccttcatgtgcgagtacgccgacgagaccgctaccatcgtggagttcctgaaccgctggatcaccttttgccagagcatcatcagcacactgaccggtggaggaggtagcgacaagacccacacttgccccccttgtcccgctccggaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggaggaggtagccaagtgcagctgcagcagcccggcgccgagctggtgagacccggcgcctccgtgaagctgagctgcaaagctagcggctacaccttcacctcctactggatcaactgggtgaagcagagacccggccaaggactggagtggatcggcaacatctacccctccgactcctacaccaactacaaccagaagtttaaggacaaggccacactgaccgtggacaagtcctccagcaccgcctacatgcagctgtcctcccctacctccgaggacagcgccgtgtactactgcgctagagtctactacggcaacagcttcgcctactggggccaaggcaccacactgaccgtgagcagcgccagcactaaggggccctctgtgtttccactcgccccttctagcaaaagcacttccggaggaactgccgctctgggctgtctggtgaaagattacttccccgaaccagtcactgtgtcatggaactctggagcactgacatctggagttcacacctttcctgctgtgctgcagagttctggactgtactccctgtcatctgtggtcaccgtgccatcttcatctctggggacccagacctacatctgtaacgtgaaccacaaaccctccaacacaaaagtggacaaacgagtcgaaccaaaatcttgt

(19) United States
(12) Patent Application Publication
(10) Pub. No.: US 2023/0303650 A1
(43) Pub. Date: Sep. 28, 2023

(54) Immunoconjugates of Interlukin-2 Mutant Polypeptides with Antibodies

(71) Applicant: Aetio Biotherapy, Inc., Dallas, TX (US)

(72) Inventors: Tao Yue, Coppell, TX (US); Yang Wang, Dallas, TX (US)

(21) Appl. No.: 18/157,264

(22) Filed: Jan. 20, 2023

Related U.S. Application Data
(60) Provisional application No. 63/301,688, filed on Jan. 21, 2022.

(51) Int. Cl. C07K 14/55; C12N 15/63

(52) U.S. Cl. CPC C07K 14/55; C12N 15/63; C07K 2319/30

(57) ABSTRACT

The present invention includes compositions, methods, nucleic acids, vectors and host cells, that include an IL-2 receptor binding fusion protein comprising: an IL-2 mutant protein that binds the IL-2 receptor; and an antibody Fc fragment; wherein IL-2 mutant protein binds and maintains its activity through the IL-2 receptor at a low pH.

### CROSS-REFERENCE TO RELATED APPLICATIONS

This application claims priority to U.S. Provisional Application Serial No. 63/301,688, filed Jan. 21, 2022, the entire contents of which are incorporated herein by reference.

### TECHNICAL FIELD OF THE INVENTION

The present invention relates in general to the field of immunotherapy, and more particularly, to novel compositions and methods of producing the mutant IL-2 polypeptides or immunoconjugates, pharmaceutical compositions comprising the same, and uses thereof.

### STATEMENT OF FEDERALLY-FUNDED RESEARCH

Not applicable.

### INCORPORATION-BY-REFERENCE OF MATERIALS FILED ON COMPACT DISC

The Sequence Listing in an XML file, named as AEBI1007.xml of 62.8 KB, created on Jan. 20, 2023 and submitted to the United States Patent and Trademark Office via Patent Center, is incorporated herein by reference.

### BACKGROUND OF THE INVENTION

Without limiting the scope of the invention, its background is described in connection with mutant IL-2 polypeptides.

In one example, US2021/0079055A1, entitled “SUPERAGONISTS, PARTIAL AGONISTS AND ANTAGONISTS OF INTERLEUKIN-2”, is said to teach IL-2 muteins that have an increased binding capacity for IL-2Rβ receptor and a decreased binding capacity for IL-2Rγc receptor, as compared to wild-type IL-2. Such IL-2 muteins are useful, for example, as IL-2 partial agonist and antagonists in applications where reduction or inhibition of one or more IL-2 and/or IL-15 functions is useful (e.g., in the treatment of graft versus host disease (GVHD) and adult T cell leukemia).

Another example is, WO 2012/107417Al entitled “MUTANT INTERLEUKIN-2 POLYPEPTIDES”, is said to teach mutant interleukin-2 polypeptides that exhibit reduced affinity to the α-subunit of the IL-2 receptor, for use as immunotherapeutic agents. In addition, the invention relates to immunoconjugates comprising said mutant IL-2 polypeptides, polynucleotide molecules encoding the mutant IL-2 polypeptides or immunoconjugates, and vectors and host cells comprising such polynucleotide molecules. The invention further relates to methods for producing the mutant IL-2 polypeptides or immunoconjugates, pharmaceutical compositions comprising the same, and uses thereof.

Another example is from Kuziel, et al., entitled, “Unexpected effects of the IL-2 receptor alpha subunit on high-affinity IL-2 receptor assembly and function detected with a mutant IL-2 analog”, J. Immunol Apr. 15, 1993, 150 (8) 3357-3365. In this publication, it was found that an F42A mutation markedly reduced the intrinsic affinity of the resultant IL-2 analog for the low-affinity IL-2Ra subunit although having little or no effect on binding to the IL-2R beta or gamma affinity receptor. The study indicated that Il-2-mediated side-effects, such as pulmonary edema, were abrogated by a blocking antibody to IL-2Ra.

Thus, a need exists for novel constructs that help target cancers.

### SUMMARY OF THE INVENTION

As embodied and broadly described herein, an aspect of the present disclosure relates to an IL-2 receptor binding fusion protein comprising: an IL-2 mutant protein that binds an IL-2 Receptor; and an antibody Fc fragment; wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4. In one aspect, the fusion protein further comprises a tumor-targeting antibody or a tumor-specific protein binding domain at a carboxy- or amino-terminus. In another aspect, the IL-2 mutant has a higher affinity for the IL-2 Receptor at pH 6.9 than IL-2. In another aspect, the IL-2 mutant has a higher affinity for the IL-2 Receptor at pH 6.4. In another aspect, the IL-2 mutant has an F42A mutation that eliminated binding to IL-2 Receptor alpha. In another aspect, a tumor antigen targeted is selected from HER1, HER2, HER3, GD2, carcinoembryonic antigens (CEAs), epidermal growth factor receptor active mutant (EGFRVIII), CD133, Fibroblast Activation Protein Alpha (FAP), Epithelial cell adhesion molecular (Epcam), Glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), or a Claudin family protein. In another aspect, a tumor is selected from the group consisting of: acute myeloid leukemia, adrenocortical carcinoma, B-cell lymphoma, bladder urothelial carcinoma, breast ductal carcinoma, breast lobular carcinoma, esophageal carcinomas, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin’s lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma. In another aspect, the fusion protein comprises human sequences. In another aspect, the fusion protein comprises, in order from amino to carboxy, the IL-2 mutant protein and the antibody Fc fragment. In another aspect, the fusion protein comprises, in order from amino to carboxy, the antibody Fc fragment and the IL-2 mutant protein. In another aspect, the fusion protein further comprises one or more protein domains at an amino-, a carboxy-, or both the amino- and carboxy-terminus of the fusion protein, wherein the one or more protein domains is an antibody, binding fragments thereof, Fc region, cytokine or receptor. In another aspect, the Fc is a wild-type or a mutated Fc. In another aspect, the fusion protein has at least 90, 91, 92, 93, 94, 95, 96, 97, 98, 99, or 100% sequence identity with SEQ ID NO:13, 14, 15, 16, or 17.

As embodied and broadly described herein, an aspect of the present disclosure relates to a method of treating a cancer patient providing a subject in need thereof an IL-2 receptor binding fusion protein comprising: providing the cancer patient with an effective amount of the IL-2 receptor binding fusion protein comprising: an IL-2 mutant protein that binds the IL-2 receptor; and an antibody Fc fragment; wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4. In one aspect, the method further comprises a tumor-targeting antibody or a tumor-specific protein binding domain at the carboxy- or amino terminus. In another aspect, the IL-2 mutant has a higher affinity for the IL-2 Receptor at pH 6.9 than IL-2. In another aspect, the IL-2 mutant has a higher affinity for the IL-2 Receptor at pH 6.4. In another aspect, the IL-2 mutant has an F42A mutation that eliminated binding to IL-2 Receptor alpha. In another aspect, a tumor antigen targeted is selected from HER1, HER2, HER3, GD2, carcinoembryonic antigens (CEAs), epidermal growth factor receptor active mutant (EGFRVIII), CD133, Fibroblast Activation Protein Alpha (FAP), Epithelial cell adhesion molecular (Epcam), Glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), or a Claudin family protein. In another aspect, a tumor is selected from the group consisting of: acute myeloid leukemia, adrenocortical carcinoma, B-cell lymphoma, bladder urothelial carcinoma, breast ductal carcinoma, breast lobular carcinoma, esophageal carcinomas, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin’s lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma. In another aspect, the fusion protein comprises human sequences. In another aspect, the fusion protein comprises, in order from amino to carboxy, the IL-2 mutant protein and the antibody Fc fragment. In another aspect, the fusion protein comprises, in order from amino to carboxy, the antibody Fc fragment and the IL-2 mutant protein. In another aspect, the method further comprises one or more protein domains at an amino-, a carboxy-, or both the amino- and carboxy-terminus of the fusion protein, wherein the one or more protein domains is an antibody, binding fragments thereof, Fc region, cytokine or receptor. In another aspect, the Fc is a wild-type or a mutated Fc. In another aspect, the fusion protein has SEQ ID NOS: 3 or 4. In another aspect, the fusion protein has at least 90, 91, 92, 93, 94, 95, 96, 97, 98, 99, or 100% sequence identity with SEQ ID NO:13, 14, 15, 16, or 17.

As embodied and broadly described herein, an aspect of the present disclosure relates to a nucleic acid that encodes an IL-2 receptor binding fusion protein comprising: an IL-2 mutant protein that binds the IL-2 receptor; and an antibody Fc fragment; wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4. In another aspect, the nucleic acid further comprises a tumor-targeting antibody or a tumor-specific protein binding domain at a carboxy- or amino-terminus. In another aspect, the L-2 mutant has a higher affinity for the IL-2 Receptor at pH 6.9 than IL-2. In another aspect, the IL-2 mutant has a higher affinity for the IL-2 Receptor at pH 6.4. In another aspect, the IL-2 mutant has an F42A mutation that eliminated binding to IL-2 Receptor alpha. In another aspect, a tumor antigen targeted is selected from HER1, HER2, HER3, GD2, carcinoembryonic antigens (CEAs), epidermal growth factor receptor active mutant (EGFRVIII), CD133, Fibroblast Activation Protein Alpha (FAP), Epithelial cell adhesion molecular (Epcam), Glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), or a Claudin family protein. In another aspect, a tumor is selected from the group consisting of: acute myeloid leukemia, adrenocortical carcinoma, B-cell lymphoma, bladder urothelial carcinoma, breast ductal carcinoma, breast lobular carcinoma, esophageal carcinomas, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin’s lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma. In another aspect, the fusion protein comprises human sequences. In another aspect, the fusion protein comprises, in order from amino to carboxy, the IL-2 mutant protein and the antibody Fc fragment. In another aspect, the fusion protein comprises, in order from amino to carboxy, the antibody Fc fragment and the IL-2 mutant protein. In another aspect, the nucleic acid further comprises one or more protein domains at an amino-, a carboxy-, or both the amino- and carboxy-terminus of the fusion protein, wherein the one or more protein domains is an antibody, binding fragments thereof, Fc region, cytokine or receptor. In another aspect, the Fc is a wild-type or a mutated Fc. In another aspect, the nucleic acid encodes the fusion protein with has at least 90, 95, 96, 97, 98, 99, or 100% sequence identity to SEQ ID NOS: 3 or 4. In another aspect, the fusion protein has at least 90, 91, 92, 93, 94, 95, 96, 97, 98, 99, or 100% sequence identity with SEQ ID NO:13, 14, 15, 16, or 17.

As embodied and broadly described herein, an aspect of the present disclosure relates to a vector that expresses a nucleic acid that encodes an IL-2 receptor binding fusion protein comprising: an IL-2 mutant protein that binds the IL-2 receptor; and an antibody Fc fragment; wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4. In another aspect, the fusion protein has at least 90, 91, 92, 93, 94, 95, 96, 97, 98, 99, or 100% sequence identity with SEQ ID NO:13, 14, 15, 16, or 17.

As embodied and broadly described herein, an aspect of the present disclosure relates to a host cell that comprises a vector that expresses a nucleic acid that encodes an IL-2 receptor binding fusion protein comprising: an IL-2 mutant protein that binds the IL-2 receptor; and an antibody Fc fragment; wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4. In another aspect, the fusion protein has at least 90, 91, 92, 93, 94, 95, 96, 97, 98, 99, or 100% sequence identity with SEQ ID NO:13, 14, 15, 16, or 17.

As embodied and broadly described herein, an aspect of the present disclosure relates to a method of making an IL-2 receptor binding fusion protein comprising: expressing a nucleic acid that encodes the IL-2 receptor binding fusion protein comprising: an IL-2 mutant protein that binds the IL-2 receptor; and an antibody Fc fragment; wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4.

As embodied and broadly described herein, an aspect of the present disclosure relates to an IL-2 receptor binding fusion protein comprising: an IL-2 mutant protein that binds the IL-2 receptor; and an antibody Fc fragment; wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4. In another aspect, the fusion protein has at least 90, 91, 92, 93, 94, 95, 96, 97, 98, 99, or 100% sequence identity with SEQ ID NO:13, 14, 15, 16, or 17.

As embodied and broadly described herein, an aspect of the present disclosure relates to a method of treating a cancer patient providing a subject in need thereof with an IL-2 receptor binding fusion protein comprising: providing the cancer patient with an effective amount of: an IL-2 mutant protein that binds the IL-2 receptor; and an antibody Fc fragment; wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4. In another aspect, the fusion protein has at least 90, 91, 92, 93, 94, 95, 96, 97, 98, 99, or 100% sequence identity with SEQ ID NO:13, 14, 15, 16, or 17.

As embodied and broadly described herein, an aspect of the present disclosure relates to a nucleic acid that encodes an IL-2 receptor binding fusion protein comprising: an IL-2 mutant protein that binds the IL-2 receptor; and an antibody Fc fragment; wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4. In one aspect, the nucleic acid has at least 90, 95, 96, 97, 98, 99, or 100% sequence identity to SEQ ID NOS: 20, 21, 30, 31, 32, 33, or 34.

As embodied and broadly described herein, an aspect of the present disclosure relates to a vector that expresses a nucleic acid that encodes an IL-2 receptor binding fusion protein comprising: an IL-2 mutant protein that binds the IL-2 receptor; and an antibody Fc fragment; wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4. In one aspect, the nucleic acid has at least 90, 95, 96, 97, 98, 99, or 100% sequence identity to SEQ ID NOS: 20, 21, 30, 31, 32, 33, or 34.

As embodied and broadly described herein, an aspect of the present disclosure relates to a host cell that comprises a vector that expresses a nucleic acid that encodes an IL-2 receptor binding fusion protein comprising: an IL-2 mutant protein that binds the IL-2 receptor; and an antibody Fc fragment; wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4. In one aspect, the nucleic acid has at least 90, 95, 96, 97, 98, 99, or 100% sequence identity to SEQ ID NOS: 20, 21, 30, 31, 32, 33, or 34.

As embodied and broadly described herein, an aspect of the present disclosure relates to a method of making an IL-2 receptor binding fusion protein comprising: expressing a nucleic acid that encodes the IL-2 receptor binding fusion protein comprising: an IL-2 mutant protein that binds the IL-2 receptor; and an antibody Fc fragment; wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4. In one aspect, the nucleic acid has at least 90, 95, 96, 97, 98, 99, or 100% sequence identity to SEQ ID NOS: 20, 21, 30, 31, 32, 33, or 34.

### BRIEF DESCRIPTION OF THE DRAWINGS

For a more complete understanding of the features and advantages of the present invention, reference is now made to the detailed description of the invention along with the accompanying figures and in which:

FIGS. 1A to 1F show: FIG. 1A shows a schematic diagram of an amino-terminus IL-2 variant (mutant) of the present invention and Fc wild-type (WT) (e.g., human). FIG. 1B shows a schematic diagram of an amino-terminus Fc WT (e.g., human) and IL-2 variant (mutant) of the present invention. FIG. 1C shows a schematic diagram of an amino-terminus IL-2 variant (mutant) of the present invention, Fc WT (e.g., human), and a target binding polypeptide (TBP). FIG. 1D shows a schematic diagram of an amino-terminus target binding polypeptide (TBP), Fc WT (e.g., human), and the IL-2 variant (mutant) of the present invention. FIG. 1E shows a heterodimer protein that has: a first polypeptide with an amino-terminus IL-2 variant (mutant) of the present invention and Fc wild-type (WT) (e.g., human) in one chain, and a second polypeptide with an amino-terminus target binding polypeptide (TBP), Fc WT (e.g., human). FIG. 1F is a heterodimer of a first polypeptide with an amino-terminus Fc WT (e.g., human) and IL-2 variant (mutant) of the present invention, with a second polypeptide with an amino-terminus Fc WT and a target binding polypeptide (TBP). A target binding polypeptide can be a tumor-targeting antibody or a tumor-specific protein-binding domain.

FIGS. 2A and 2B show the selection and identification of pH-resistant IL2. Human IL-2 variants (muteins), identified by our yeast surface display screen, exhibit stronger binding affinity to human IL-2R beta at pH 6.9 than WT protein. FIG. 2A is flow cytometry data shows that IL-2R beta at a series of concentrations, binds to yeast expressing IL-2 mutein more strongly than IL-2 WT at pH6.9. FIG. 2B is ELISA data shows that IL-2R beta binds to IL-2 mutein, but not WT at pH 6.4, when both contains IL-2 mutein and WT contains F42A mutation to eliminate the binding to IL-2R alpha.

FIG. 3 shows CFSE-labeled human PBMCs were treated with 10 nM IL-2 WT or variants for 6 days. The proliferation of CD8+ T cells was analyzed by flow cytometry.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The novel IL-2 variants (muteins) of the present invention contain R81E and L85I or L85T (L85I/T) mutations, which are not reported in any published patents or prior art. The IL-2 variants of the present invention exhibit increased binding affinities to IL-2 receptor beta at a low pH and increased activities to promote CD8+ T cell proliferation, as compared to wild-type IL-2. These increased binding affinities at the low pH are not mentioned in any mutants reported.

F42A mutation markedly reduces IL-2 binding for IL-2R alpha, but not beta or gamma, leading to dampened side-effects from a low-dose IL-2 analog treatment. Some of the IL-2 variants contain three-residue substitutions including F42A, R81E, and L85I/T, and the resultant variants including three-residue substitutions have significantly increased binding affinity at the low pH and even lower side-effect compared with F42A mutation alone.

As used herein, the term “IL-2 variant binds the IL-2 Receptor at low pH” refers to an IL-2 variant or mutein that has a high binding affinity for the IL-2 Receptor at a pH below 7. In certain aspects, the IL-2 variant has a higher binding affinity for the IL-2 Receptor at pH of 6.9, 6.8, 6.7, 6.6, 6.5, 6.4, 6.3, 6.2, 6.1, or 6.0, when compared to wild-type or non-mutated IL-2. In certain embodiments, the IL-2 Receptor is the IL-2 Receptor beta. The IL-2 variant binds the IL-2 Receptor at low pH, specifically, the tumor microenvironment. Examples of IL-2 variant binds the IL-2 Receptor at low pH include those of SEQ ID NOS: 3 and 4.

In some immunoconjugates, antibodies against tumor-specific antigens, tumor-specific antigens, or T cell-associated antigens are fused to the novel IL-2 variants and enrich them in the tumor tissues to better activate T cells in the tumor microenvironment for tumor elimination. The present invention not only increases the anti-tumor effect but also further reduces the side effects. Plus, the fusion of IL-2 variants to antibodies is significantly extending the half-life of the cytokines.

Therapeutic application of these immunoconjugates allows patients to tolerate IL-2 treatment without strong side effects and benefit from cancer therapy, and in some cases prevent death from unintended immune effects caused by immunotherapies.

High-dose Interleukin-2 (IL-2), used in the treatment of cancers, is associated with significant side effects due to its toxicity in multiple organ systems, including the heart, lungs, kidneys, and central nervous system. IL-2 has shown a reduced binding-affinity for IL-2 receptor beta at a low pH, and this acidic pH often occurs in the tumor microenvironment due to glycolysis in tumor cells and insufficient blood perfusion. Therefore, it was critical to increase IL-2 binding affinity for its receptor and maintain its activity at the low pH for improved IL-2-mediated antitumor effect so that a relatively lower dose IL-2 can be used to treat cancer patients with weak side effects. The inventors engineered novel IL-2 mutant polypeptides that are exhibiting increased binding affinities for IL-2 receptor beta at the low pH, and higher activities to promote CD8+ T cell proliferation, as compared with wild-type IL-2. In addition, the invention relates to immunoconjugates comprising said mutant IL-2 polypeptides, antibodies, and other previously generated mutations. The invention further relates to methods for producing the mutant IL-2 polypeptides or immunoconjugates, pharmaceutical compositions comprising the same, and uses thereof.

As used herein, the term “antibody” is used in the broadest sense, and specifically covers monoclonal antibodies (including full length antibodies or other bivalent, Fc-region containing antibodies such as bivalent scFv Fc-fusion antibodies), polyclonal antibodies, multispecific antibodies (e.g., bispecific antibodies), and antibody fragments (e.g., Fab, Fab′, F(ab′)₂, Fv, scFv) so long as they exhibit the desired biological activity. An intact antibody or a binding fragment thereof, competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain variable fragment (scFv) antibodies. An antibody other than a “bispecific” or “bifunctional” antibody is understood to have identical binding sites. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay). Antibodies (Abs) and immunoglobulins (Igs) are glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas. The present invention includes monoclonal antibodies (and binding fragments thereof) that are completely recombinant, in other words, where the complementarity determining regions (CDRs) are genetically spliced into a human antibody backbone, often referred to as veneering an antibody. Thus, in certain aspects, the monoclonal antibody is a fully synthesized antibody. In certain embodiments, the monoclonal antibodies (and binding fragments thereof) can be made in bacterial or eukaryotic cells, including plant cells.

As used herein, the term “antibody binding fragment” refers to a portion of a full-length antibody, generally the antigen binding or variable region and include Fab, Fab′, F(ab′)₂, Fv and scFv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of cross-linking antigen, and a residual other fragment (which is termed pFc′). As used herein, “functional fragment” with respect to antibodies, refers to Fv, F(ab) and F(ab′)₂ fragments.

As used herein, the “Fv” fragment is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond. While the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985), relevant portions incorporated herein by reference.

As used herein, an “isolated” antibody is one that has been identified and separated and/or recovered from a component of the environment in which it was produced. Contaminant components of its production environment are materials, which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody’s natural environment will not be present. Ordinarily, however, isolated antibodies will be prepared by at least one purification step.

As used herein, the term “antibody mutant” refers to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95, 96, 97, 98, or 99%. In one aspect, the fusion protein has at least 90, 91, 92, 93, 94, 95, 96, 97, 98, 99, or 100% sequence identity with SEQ ID NO: 13, 14, 15, 16, or 17.

As used herein, the term “variable” in the context of variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877), or both, that is Chothia plus Kabat. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat et al.) The constant domains are not involved directly in binding an antibody to an antigen, but exhibit various effector function, such as participation of the antibody in antibody-dependent cellular toxicity.

The light chains of antibodies (immunoglobulin) from any vertebrate species can be assigned to one of two clearly distinct types, called kappa and lambda, based on the amino sequences of their constant domain.

Depending on the amino acid sequences of the constant domain of their heavy chains, “immunoglobulins” can be assigned to different classes. There are at least five (5) major classes of immunoglobulins: IgA, IgD, IgE, IgG and IgM, and several of these may be further divided into subclasses (isotypes), e.g., IgG-1, IgG-2, IgG-3 and IgG4; IgA-1 and IgA-2. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed and claimed invention may be made by the hybridoma method first described by Kohler and Milstein, Nature 256, 495 (1975), relevant portions incorporated herein by reference.

All monoclonal antibodies utilized in accordance with the presently disclosed and claimed invention will be either (1) the result of a deliberate immunization protocol, as described in more detail herein below; or (2) the result of an immune response that results in the production of antibodies naturally in the course of a disease or cancer.

The uses of the monoclonal antibodies of the presently disclosed and claimed invention may require administration of such or similar monoclonal antibody to a subject, such as a human. However, when the monoclonal antibodies are produced in a non-human animal, such as a rodent or chicken, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed and claimed invention can be “humanized”, that is, the antibodies are engineered such that antigenic portions thereof are removed and like portions of a human antibody are substituted therefore, while the antibodies’ affinity for a specific antigen is retained. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, relevant portions incorporated herein by reference.

Humanized forms of antibodies are chimeric immunoglobulins, immunoglobulin chains or fragments thereof (such as Fab, Fab′, F(ab′)₂, Fv, scFv or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting nonhuman (i.e. rodent, chicken) CDRs or CDR sequences for the corresponding sequences of a human antibody. (See also U.S. Pat. No. 5,225,539.) In some instances, Fᵥ framework residues of the human immunoglobulin are replaced by corresponding non-human residues from the donor antibody. Humanized antibodies can also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin.

As used herein, the term “bispecific” or “bifunctional” antibody is understood to have two different binding sites, one of which is an IL-2 receptor binding site and the other an antigen binding site. The antigen-binding domain can be a tumor-specific antigen, while the other antigen-binding region with bind a T cell activating molecule on a T cell.

As used herein, the term “polypeptide” refers to an amino acid chain, regardless of length or post-translational modification (e.g., glycosylation or phosphorylation). Polypeptides can include full-length proteins or fragments or variants thereof. A “polypeptide of interest” refers to a target sequence expressed at the cell, as described herein. In some embodiments, a polypeptide of interest can be a polypeptide that is not expressed in nature in the relevant type of cell or is not expressed at the level that the polypeptide is expressed when the expression is achieved by the intervention of the hand of man, as described herein. In certain embodiments, a polypeptide of interest can include sequences that are not naturally found in the relevant cell but are found naturally in other cell types or organisms.

As used herein, the term “fusion protein” refers to a hybrid protein, that includes portions of two or more different polypeptides, or fragments thereof, resulting from the expression of a polynucleotide that encodes at least a portion of each of the two polypeptides.

As used herein, the terms “nucleic acid,” “polynucleotide,” and “oligonucleotide” are used interchangeably herein to refer to a polymer of at least three nucleotides. A nucleoside comprises a nitrogenous base linked to a sugar molecule. In a polynucleotide, phosphate groups covalently link adjacent nucleosides to form a polymer. The polymer can include natural nucleosides (e.g., adenosine, thymidine, guanosine, cytidine, uridine, deoxyadenosine, deoxythymidine, deoxyguanosine, and deoxycytidine), nucleoside analogs, chemically modified bases, biologically modified bases (e.g., methylated bases), intercalated bases, and/or modified sugars (e.g., modified purines or pyrimidines). See, Kornberg and Baker (1992) DNA Replication, 2nd Ed., Freeman, San Francisco, CA; Scheit (1980) Nucleotide Analogs, John Wiley, New York, NY), and U.S. Pat. Publication No. 2004/0092470 and references therein for further discussion of various nucleotides, nucleosides, and backbone structures that can be used in the polynucleotides described herein. A polynucleotide can have any length and sequence and can be single-stranded or double-stranded. Where this document provides a nucleic acid sequence, the complementary sequence also is provided. Further, where a sequence is provided as DNA, the corresponding RNA sequence (i.e., the sequence in which T is replaced by U) also is provided.

As used herein, the term “nucleic acid construct” refers to a nucleic acid that has been recombinantly modified or is derived from such a nucleic acid. For example, a nucleic acid construct can contain a mutation, deletion, or substitution relative to a naturally occurring nucleic acid molecule. A nucleic acid construct can comprise two or more nucleic acid segments that are derived from or originate from different sources such as different organisms (e.g., a recombinant polynucleotide). The sequence of one or more portions of a nucleic acid construct may be entirely invented by man.

As used herein, the “nucleic acid sequence” refers to the nucleic acid material itself and is not restricted to the sequence information (i.e., the succession of letters chosen among the five base letters A, G, C, T, or U) that biochemically characterizes a specific nucleic acid (e.g., DNA or RNA) molecule.

As used herein, the term “gene” has its meaning as understood in the art. In general, the term “gene” refers to a nucleic acid that includes a portion encoding a protein; the term optionally may encompass regulatory sequences such as promoters, enhancers, terminators, etc., in addition to coding sequences (open reading frames). This definition is not intended to exclude application of the term “gene” to non-protein coding expression units but rather to clarify that, in most cases, the term as used in this document refers to a protein-encoding nucleic acid. It will be appreciated that the definition of gene can include nucleic acids that do not encode proteins, but rather provide templates for transcription of functional RNA molecules such as tRNAs or rRNAs, for example.

As used herein, the terms “gene product” or “expression product” refer to, in general, an RNA transcribed from the gene, or a polypeptide encoded by an RNA transcribed from the gene. Expression of a gene or a polynucleotide refers to (1) transcription of RNA from the gene or polynucleotide; (2) translation of RNA transcribed from the gene or polynucleotide, or both (1) and (2).

As used herein, the term “vector” refers to a nucleic acid or a virus, viral genome, plasmid, or portion thereof that is a nucleic acid molecule that can replication and/or express a nucleic acid molecule in cell. Where the vector is a nucleic acid, the nucleic acid molecule to be transferred is generally linked to, e.g., inserted into, the vector nucleic acid molecule. A nucleic acid vector may include sequences that direct autonomous replication within suitable host cells (e.g., an origin of replication), or may include sequences sufficient to allow integration of part of all of the nucleic acid into host cell DNA. Useful nucleic acid vectors include, for example, DNA or RNA plasmids, cosmids, and naturally occurring or modified viral genomes or portions thereof, or nucleic acids (DNA or RNA) that can be packaged into viral capsids. Plasmid vectors typically include an origin of replication and one or more selectable markers. Plasmids may include part or all of a viral genome (e.g., a viral promoter, enhancer, processing or packaging signals, etc.). Viruses or portions thereof (e.g., viral capsids) that can be used to introduce nucleic acid molecules into cells are referred to as viral vectors. Useful animal viral vectors include adenoviruses, retroviruses, lentiviruses, vaccinia virus and other poxviruses, herpes simplex virus, and others. Useful plant viral vectors include those based on to bamoviruses, ilarviruses, etc. Viral vectors may or may not contain sufficient viral genetic information for production of infectious virus when introduced into host cells, i.e., viral vectors may be replication-defective, and such replication-defective viral vectors may be preferable for certain embodiments of the invention. Where sufficient information is lacking it may, but need not be, supplied by a host cell or by another vector introduced into the cell.

As used herein, the term “expression vector” is a vector that includes one or more expression control sequences, and an “expression control sequence” is a DNA sequence that controls and regulates the transcription and/or translation of another DNA sequence.

As used herein, the term “operably linked” refers to a relationship between two nucleic acid sequences wherein the expression of one of the nucleic acid sequences is, e.g., controlled by, regulated by, or modulated by the other nucleic acid sequence. For example, transcription of a nucleic acid sequence is directed by an operably linked promoter sequence; post-transcriptional processing of a nucleic acid is directed by an operably linked processing sequence; translation of a nucleic acid sequence is directed by an operably linked translational regulatory sequence; transport or localization of a nucleic acid or polypeptide is directed by an operably linked transport or localization sequence; and post-translational processing of a polypeptide is directed by an operably linked processing sequence. A nucleic acid sequence that is operably linked to a second nucleic acid sequence typically is covalently linked, either directly or indirectly, to such a sequence, although any effective three-dimensional association is acceptable. It is noted that a single nucleic acid sequence can be operably linked to a plurality of other sequences. For example, a single promoter can direct transcription of multiple RNA species. A coding sequence is “operably linked” and “under the control” of an expression control sequence in a cell when RNA polymerase is able to transcribe the coding sequence into mRNA, which then can be translated into the protein encoded by the coding sequence.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; Fitchen, et al. (1993) Annu Rev. Microbiol. 47:739-764; Tolstoshev, et al. (1993) in Genomic Research in Molecular Medicine and Virology, Academic Press; and Ausubel et al. (1992) Current Protocols in Molecular Biology, Greene/Wiley, New York, N.Y. Abbreviations and nomenclature, where employed, are deemed standard in the field and commonly used in professional journals such as those cited herein. Many of the procedures useful for practicing the present invention, whether or not described herein in detail, are well known to those skilled in the arts of molecular biology, biochemistry, immunology, and medicine.

As used herein, the term “host cell” refers to cells into which a recombinant expression vector can be introduced. A host cell for use with the disclosed expression systems and methods typically is a eukaryotic cell, such as a mammalian, insect, or plant cell. As used herein, “transformed” and “transfected” encompass the introduction of a nucleic acid molecule (e.g., a vector) into a cell by one of a number of techniques.

As used herein, the term “identity” refers to the extent to which two or more nucleic acid sequences or two or more amino acid sequences are the same. The percent identity between two sequences over a window of evaluation is computed by aligning the sequences, determining the number of nucleotides or amino acids within the window of evaluation that are opposite an identical nucleotide or amino acid, allowing the introduction of gaps to maximize identity, dividing by the total number of nucleotides or amino acids in the window, and multiplying by 100.

Percent identity for any nucleic acid or amino acid sequence is determined as follows. First, a nucleic acid or amino acid sequence is compared to the identified nucleic acid or amino acid sequence using the BLAST 2 Sequences (Bl2seq) program from the stand-alone version of BLASTZ containing BLASTN version 2.0.14 and BLASTP version 2.0.14, or equivalents. This stand-alone version of BLASTZ can be accessed at the U.S. government’s National Center for Biotechnology Information web site (World Wide Web at ncbi.nlm.nih.gov/blast/executables). Instructions explaining how to use the Bl2seq program can be found in the readme file accompanying BLASTZ.

In some cases, the nucleic acid or polypeptide sequence can include a sequence with at least 90 percent sequence identity (e.g., at least 90, 91, 92, 93, 94, 95, 96, 97, 98, or 99 percent sequence identity, or 100 percent sequence identity) to one or more sequences as set forth in SEQ ID NO:13, 14, 15, 16, or 17, so long as the mutant IL-2 amino acid has 100% identity to SEQ ID NO: 3 or 4 (while the nucleic acid sequences can include known alternative codons, with exemplary sequences of SEQ ID NOS: 20 or 21). In some embodiments, the fusion protein can have an amino acid sequence with at least 90 percent sequence identity (e.g., at least 90, 91, 92, 93, 94, 95, 96, 97, 98, or 99 percent sequence identity, or 100 percent sequence identity) to one or more sequences disclosed herein, however, certain specific amino acid sequences are those set forth in SEQ ID NO:13, 14, 15, 16, or 17, which are nucleic acid sequences SEQ ID NO: 30, 31, 32, 33, or 34, respectively.

As used herein, the term “biologically active moiety” or “biologically active moieties” refer to a polypeptide, portion, fragment, or component of a polypeptide capable of performing a function, an action, or a reaction in a biological context. A biologically active moiety may comprise a complete protein or biologically active portion(s) thereof. For example, the term “biologically active moiety” includes active and functional molecules, binding domains of molecules that bind to components of a biological system, such as portions of a cytokine and/or cytokine receptor. Non-limiting examples of cytokines and/or their receptors that can be used with the present invention include, e.g., at least one of the biologically active moieties comprises: IL-1, IL-2, IL-4, IL-5, IL-6, IL-7, IL-8, IL-10, IL-11, IL-12, IL-13, IL-15, IL-18, IL-21, interferons α, β, or γ, colony-stimulating factors (CSFs), granulocyte-macrophage CSF, tumor necrosis factor alpha, or tumor necrosis factor beta.

The antigen-binding portion can target a cancer, which can be selected from the group consisting of: acute myeloid leukemia, adrenocortical carcinoma, B-cell lymphoma, bladder urothelial carcinoma, breast ductal carcinoma, breast lobular carcinoma, carcinomas of the esophagus, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin’s lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma. Example of tumor associates antigens (TAA) for targeting with the BiTEs of the present invention can be selected from HER1, HER2, HER3, GD2, carcinoembryonic antigens (CEAs), epidermal growth factor receptor active mutant (EGFRVIII), CD133, Fibroblast Activation Protein Alpha (FAP), Epithelial cell adhesion molecular (Epcam), Glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), or a Claudin family protein.

As used herein, the term “scFc polypeptide” refers to a polypeptide comprising a single-chain Fc (scFc) region and the IL-2 variant of the present invention.

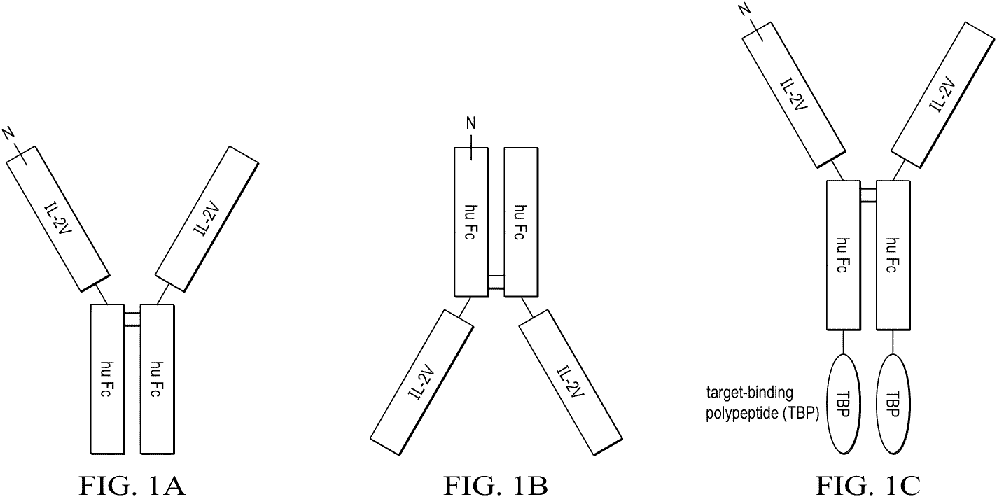

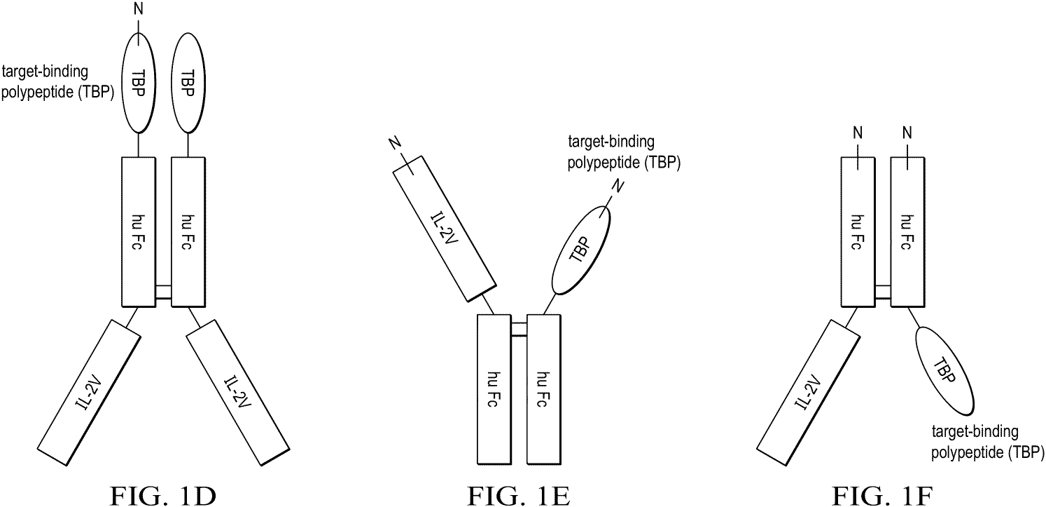

FIGS. 1A to 1F show: FIG. 1A shows a schematic diagram of an amino-terminus IL-2 variant (mutant) of the present invention and Fc wild-type (WT) (e.g., human). FIG. 1B shows a schematic diagram of an amino-terminus Fc WT (e.g., human) and IL-2 variant (mutant) of the present invention. FIG. 1C shows a schematic diagram of an amino-terminus IL-2 variant (mutant) of the present invention, Fc WT (e.g., human), and a target binding polypeptide (TBP). FIG. 1D shows a schematic diagram of an amino-terminus target binding polypeptide (TBP), Fc WT (e.g., human), and the IL-2 variant (mutant) of the present invention. FIG. 1E shows a heterodimer protein that has: a first polypeptide with an amino-terminus IL-2 variant (mutant) of the present invention and Fc wild-type (WT) (e.g., human) in one chain, and a second polypeptide with an amino-terminus target binding polypeptide (TBP), Fc WT (e.g., human). FIG. 1F is a heterodimer of a first polypeptide with an amino-terminus Fc WT (e.g., human) and IL-2 variant (mutant) of the present invention, with a second polypeptide with an amino-terminus Fc WT and a target binding polypeptide (TBP). A target binding polypeptide can be a tumor-targeting antibody or a tumor-specific protein-binding domain.

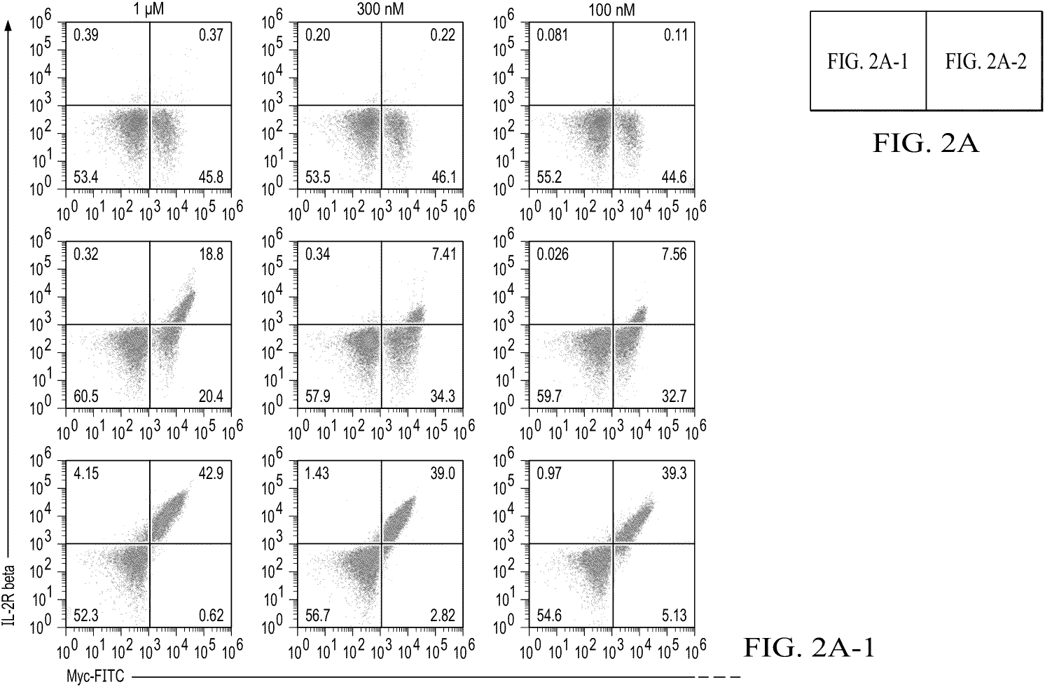

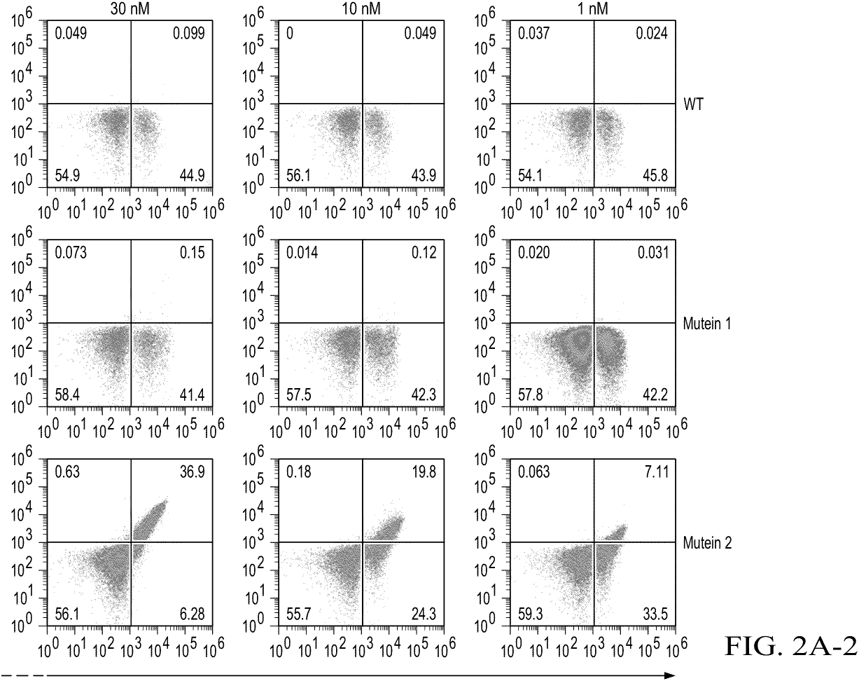

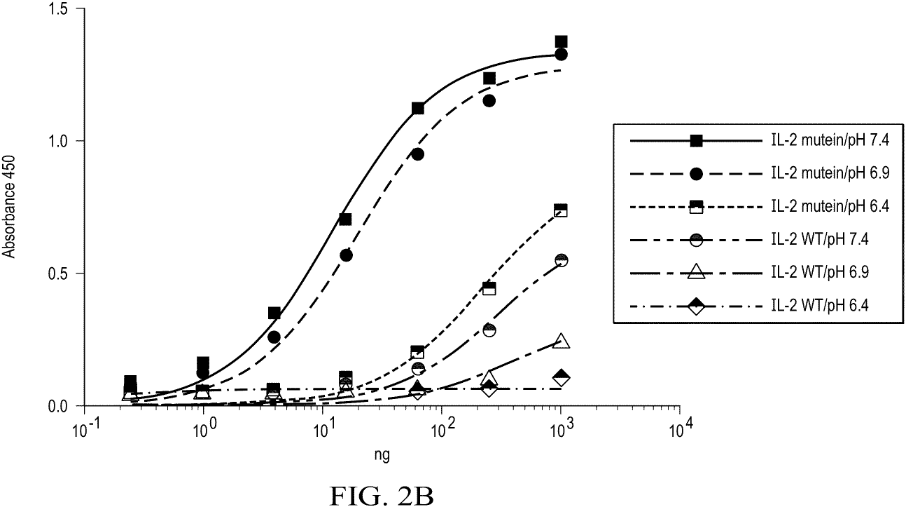

FIGS. 2A and 2B show the selection and identification of pH-resistant IL-2. Human IL-2 variants (muteins), identified by our yeast surface display screen, exhibit stronger binding affinity to human IL-2R beta at pH 6.9 than WT protein. FIG. 2A is flow cytometry data shows that IL-2R beta at a series of concentrations, binds to yeast expressing IL-2 mutein more strongly than IL-2 WT at pH 6.9. FIG. 2B is ELISA data shows that IL-2R beta binds to IL-2 mutein, but not WT at pH 6.4, when both contains IL-2 mutein and WT contains F42A mutation to eliminate the binding to IL-2R alpha.

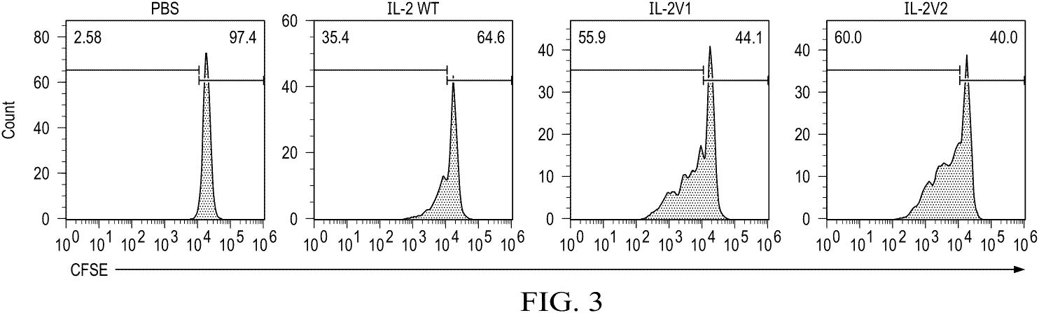

FIG. 3 shows CFSE-labeled human PBMCs were treated with 10 nM IL-2 WT or variants for 6 days. The proliferation of CD8+ T cells was analyzed by flow cytometry.

SEQ ID NO:1: human IL-2WT amino acid sequence

APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT

SEQ ID NO:18: human IL-2WT nucleic acid sequence

GCCCCTACAAGCAGCAGCACCAAGAAGACCCAGCTGCAGCTGGAACACCT
GCTGCTGGATCTGCAGATGATCCTGAACGGCATCAACAACTACAAGAACC
CCAAGCTGACCCGGATGCTGACCTTCAAGTTCTACATGCCCAAGAAGGCC
ACCGAGCTGAAGCACCTCCAGTGTCTGGAGGAGGAGCTGAAGCCTCTGGA
GGAAGTGCTGAACCTGGCCCAGAGCAAGAACTTCCACTTAAGACCCAGGG
ACTTAATCTCCAACATCAACGTGATAGTGCTGGAACTGAAGGGCAGCGAG
ACCACCTTCATGTGCGAGTACGCCGACGAGACCGCTACCATCGTGGAGTT
CCTGAACCGCTGGATCACCTTTTGCCAGAGCATCATCAGCACACTGACC

SEQ ID NO:2: human IL-2 mutant F42A

APTSSSTKKTQLQLEHLLLDLQMILNGINNYKNPKLTRMLTAKFYMPKKA
TEKHLQCLEEELKPLEEVLNLAQSKNFHLRPRDLISNINVIVLELKGSET
TFMCEYADETATIVEFLNRWITFCQSIISTLT

SEQ ID NO:19: human IL-2 mutant F42A

GCCCCTACAAGCAGCAGCACCAAGAAGACCCAGCTGCAGCTGGAACACCT
GCTGCTGGATCTGCAGATGATCCTGAACGGCATCAACAACTACAAGAACC
CCAAGCTGACCCGGATGCTGACCGCCAAGTTCTACATGCCCAAGAAGGCC
ACCGAGCTGAAGCACCTCCAGTGTCTGGAGGAGGAGCTGAAGCCTCTGGA
GGAAGTGCTGAACCTGGCCCAGAGCAAGAACTTCCACTTAAGACCCAGGG
ACTTAATCTCCAACATCAACGTGATAGTGCTGGAACTGAAGGGCAGCGAG
ACCACCTTCATGTGCGAGTACGCCGACGAGACCGCTACCATCGTGGAGTT
CCTGAACCGCTGGATCACCTTTTGCCAGAGCATCATCAGCACACTGACC

SEQ ID NO:3: human IL-2 mutant F42AV1

APTSSSTKKTQLQLEHLLLDLQMILNGINNYKNPKLTRMLTAKFYMPKKA
TELKHLQCLEEELKPLEEVLNLAQSKNFHLEPRDIISNINVIVLELKGSE
TTFMCEYADETATIVEFLNRWITFCQSIISTLT

SEQ ID NO:20: human IL-2 mutant F42AV1 (in bold)

GCCCCTACAAGCAGCAGCACCAAGAAGACCCAGCTGCAGCTGGAACACCT
GCTGCTGGATCTGCAGATGATCCTGAACGGCATCAACAACTACAAGAACC
CCAAGCTGACCCGGATGCTGACCGCCAAGTTCTACATGCCCAAGAAGGCC
ACCGAGCTGAAGCACCTCCAGTGTCTGGAGGAGGAGCTGAAGCCTCTGGA
GGAAGTGCTGAACCTGGCCCAGAGCAAGAACTTCCACTTAGAACCCAGGG
ACATAATCTCCAACATCAACGTGATAGTGCTGGAACTGAAGGGCAGCGAG
ACCACCTTCATGTGCGAGTACGCCGACGAGACCGCTACCATCGTGGAGTT
CCTGAACCGCTGGATCACCTTTTGCCAGAGCATCATCAGCACACTGACC

SEQ ID NO:4: human IL-2 mutant F42AV2

APTSSSTKKTQLQLEHLLLDLQMILNGINNYKNPKLTRMLTAKFYMPKKA
TELKHLQCLEEELKPLEEVLNLAQSKNFHLEPRDTISNINVIVLELKGSE
TTFMCEYADETATIVEFLNRWITFCQSIISTLT

SEQ ID NO:21: human IL-2 mutant F42AV2 (in bold)

GCCCCTACAAGCAGCAGCACCAAGAAGACCCAGCTGCAGCTGGAACACCT
GCTGCTGGATCTGCAGATGATCCTGAACGGCATCAACAACTACAAGAACC
CCAAGCTGACCCGGATGCTGACCGCCAAGTTCTACATGCCCAAGAAGGCC
ACCGAGCTGAAGCACCTCCAGTGTCTGGAGGAGGAGCTGAAGCCTCTGGA
GGAAGTGCTGAACCTGGCCCAGAGCAAGAACTTCCACTTAGAACCCAGGG ACACAATCTCCAACATCAACGTGATAGTGCTGGAACTGAAGGGCAGCGAG
ACCACCTTCATGTGCGAGTACGCCGACGAGACCGCTACCATCGTGGAGTT
CCTGAACCGCTGGATCACCTTTTGCCAGAGCATCATCAGCACACTGACC

The present invention differs from the art of US20210079055A1, because in the present invention: (1) the IL-2 muteins of the present invention are contain R81E, and L85I/T mutations, which are not reported in their patent; (2) the IL-2 variants of the present invention exhibit an increased binding affinity to IL-2 receptor beta at a low pH and an increased activity to promote CD8+ T cell proliferation, as compared to wild-type IL-2. An increased binding affinity at the low pH is not mentioned in any mutants reported in their patent, and other published patents. The present inventors have found that it is critical to increase the compromised binding affinity between IL-2 and its receptor caused by the acidic tumor environment to improve the anti-tumor effect of the IL-2 variant when used in the treatment of cancers; (3) besides R81E, and L85I /T mutations, some of the IL-2 variants also harbor a substitution of F42A. The F42A mutation has been shown to markedly reduce the binding affinity of the corresponding IL-2 variant for IL-2R alpha, but not beta or gamma, and greatly reduced high-dose of IL-2-induced side effects in multiple organs. By combining F42A with the IL-2 muteins of the present invention (R81E and L85I/T), the resultant IL-2 variants show stronger activity in tumor microenvironment, which lead to reduced side effects in other organs; finally, (4) antibodies against tumor-specific antigens, tumor-specific antigens or T cell-associated antigens were fused to the novel IL-2 mutein, which enriched them in the tumor tissues to better activate T cells in tumor microenvironment for the tumor elimination.

The present invention differs from the art of WO2012/107417Al, because in the present invention: (1) the IL-2 variants of the present invention contain R81E and L85I/T mutations to achieve increased binding affinities for IL-2 receptor beta at a low pH and increased activities to promote CD8+ T cell proliferation (as compared to wild-type IL-2). In contrast, the muteins listed in WO2012/107417Al do not show these activities; (2) to further lower the side effect of IL-2 muteins, F42A, a previously reported mutation, can be/was combined with R81E and L85I/T, to reduce off-target effects of IL-2 variants.

The present invention differs from the art of Kuziel, et al., J Immunol Apr. 15, 1993, 150 (8) 3357-3365, because in the present invention: (1) the inventor engineered novel IL-2 mutations to increase both the binding affinities and activities of the resultant IL-2 variants, leading to reduced dose of IL-2 analog used for effective cancer treatment and weak side-effect. R81E and L85I/T mutations of the present invention were further combined with F42A to generate IL-2 variants with higher activity at a low pH and lower side-effect, and the resultant variants including three-residue substitutions that significantly increased binding affinity at the low pH and even lower side-effect compared with F42A mutation alone.

SEQ ID NO:5: human IgG1 Fc (human Fc, dimeric, wild-type)

DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK

SEQ ID NO:22: human IgG1 Fc (human Fc, dimeric, wild-type)

GACAAGACCCACACTTGCCCCCCTTGTCCCGCTCCGGAACTCCTGGGCGG
ACCGTCAGTCTTCCTCTTCCCCCCAAAACCCAAGGACACCCTCATGATCT
CCCGGACCCCTGAGGTCACATGCGTGGTGGTGGACGTGAGCCACGAAGAC
CCTGAGGTCAAGTTCAACTGGTACGTGGACGGCGTGGAGGTGCATAATGC
CAAGACAAAGCCGCGGGAGGAGCAGTACAACAGCACGTACCGTGTGGTCA
GCGTCCTCACCGTCCTGCACCAGGACTGGCTGAATGGCAAGGAGTACAAG
TGCAAGGTCTCCAACAAAGCCCTCCCAGCCCCCATCGAGAAAACCATCTC
CAAAGCCAAAGGGCAGCCCCGAGAACCACAGGTGTACACCCTGCCCCCAT
CCCGGGATGAGCTGACCAAGAACCAGGTCAGCCTGACCTGCCTGGTCAAA
GGCTTCTATCCCAGCGACATCGCCGTGGAGTGGGAGAGCAATGGGCAGCC
GGAGAACAACTACAAGACCACGCCTCCCGTGTTGGACTCCGACGGCTCCT
TCTTCCTCTACAGCAAGCTCACCGTGGACAAGAGCAGGTGGCAGCAGGGG
AACGTCTTCTCATGCTCCGTGATGCATGAGGCTCTGCACAACCACTACAC
GCAGAAGAGCCTCTCCCTGTCTCCGGGTAAA

SEQ ID NO:6: human IgG1 Fc (human Fc, dimeric, mutant)

DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALGAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK

SEQ ID NO:23: human IgG1 Fc (human Fc, dimeric, mutant)

GACAAAACTCACACATGCCCACCGTGCCCAGCACCGGAAGCCGCAGGCGG
ACCGTCAGTCTTCCTCTTCCCCCCAAAACCCAAGGACACCCTCATGATCT
CCCGGACCCCTGAGGTCACATGCGTGGTGGTGGACGTGAGCCACGAAGAC
CCTGAGGTCAAGTTCAACTGGTACGTGGACGGCGTGGAGGTGCATAATGC
CAAGACAAAGCCGCGGGAGGAGCAGTACAACAGCACGTACCGTGTGGTCA
GCGTCCTCACCGTCCTGCACCAGGACTGGCTGAATGGCAAGGAGTACAAG
TGCAAGGTCTCCAACAAAGCCCTCGGAGCCCCCATCGAGAAAACCATCTC
CAAAGCCAAAGGGCAGCCCCGAGAACCACAGGTGTACACCCTGCCCCCAT
CCCGGGATGAGCTGACCAAGAACCAGGTCAGCCTGACCTGCCTGGTCAAA
GGCTTCTATCCCAGCGACATCGCCGTGGAGTGGGAGAGCAATGGGCAGCC
GGAGAACAACTACAAGACCACGCCTCCCGTGTTGGACTCCGACGGCTCCT
TCTTCCTCTACAGCAAGCTCACCGTGGACAAGAGCAGGTGGCAGCAGGGG
AACGTCTTCTCATGCTCCGTGATGCATGAGGCTCTGCACAACCACTACAC
GCAGAAGAGCCTCTCCCTGTCTCCGGGTAAA

SEQ ID NO:7: human IgG1 Fc (human Fc, monomeric, wild-type)

APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTKPPSRDELTKNQVSLSCLVKGFYPSDIAVE
WESNGQPENNYKTTVPVLDSDGSFRLASYLTVDKSRWQQGNVFSCSVMHE
ALHNHYTQKSLSLSPGK

SEQ ID NO:24: human IgG1 Fc (human Fc, monomeric, wild-type)

GCGCCGGAGCTGCTCGGTGGACCATCAGTATTCCTCTTCCCACCGAAGCC
TAAGGATACTCTCATGATATCCAGGACTCCTGAGGTTACTTGTGTTGTGG
TTGACGTATCTCATGAGGACCCTGAGGTCAAATTTAATTGGTATGTAGAC
GGAGTGGAAGTCCACAATGCCAAGACTAAACCAAGGGAGGAGCAGTATAA
CTCTACCTACCGCGTTGTGTCAGTGCTGACGGTGTTGCATCAAGACTGGC
TGAACGGGAAGGAATACAAATGTAAGGTGTCCAACAAGGCCCTCCCTGCG
CCTATAGAAAAAACTATAAGTAAAGCCAAAGGTCAACCAAGAGAACCTCA
GGTATATACGAAACCACCCTCCAGGGACGAGCTTACCAAGAACCAGGTTT
CACTGAGTTGTCTCGTCAAGGGGTTCTACCCAAGCGATATTGCGGTCGAG
TGGGAGTCTAATGGACAACCGGAGAATAACTACAAAACTACCGTACCTGT
GCTGGATAGCGATGGGTCTTTCAGACTCGCCTCTTACTTGACGGTCGATA
AAAGTCGATGGCAGCAGGGAAACGTCTTTAGCTGCAGTGTCATGCATGAA
GCACTCCACAACCATTACACTCAGAAAAGTCTTTCTCTGAGTCCAGGTAA
G

SEQ ID NO:8: human IgG1 Fc (human Fc, monomeric, mutant)

APEAAGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALGA
PIEKTISKAKGQPREPQVYTKPPSRDELTKNQVSLSCLVKGFYPSDIAVE
WESNGQPENNYKTTVPVLDSDGSFRLASYLTVDKSRWQQGNVFSCSVMHE
ALHNHYTQKSLSLSPGK

SEQ ID NO:25: human IgG1 Fc (human Fc, monomeric, mutant)

GCCCCGGAAGCAGCTGGCGGCCCATCCGTATTCCTTTTTCCTCCCAAACC
CAAGGACACGCTGATGATTAGCCGAACACCGGAGGTGACGTGCGTTGTTG
TGGATGTGAGCCACGAAGACCCAGAGGTCAAGTTTAATTGGTACGTAGAC
GGCGTTGAGGTACATAATGCCAAGACGAAACCTAGAGAGGAGCAGTATAA
CTCTACATATCGAGTGGTTTCAGTGCTTACTGTTTTGCATCAGGATTGGT
TGAATGGTAAGGAATACAAGTGTAAAGTGTCAAACAAAGCACTGGGGGCA
CCGATAGAAAAAACTATCAGTAAGGCAAAAGGACAACCGCGAGAACCACA
AGTATATACAAAACCCCCTTCCCGAGACGAACTTACTAAGAACCAAGTAA
GTCTGTCCTGTTTGGTAAAAGGCTTTTACCCCTCAGACATAGCCGTAGAG
TGGGAATCTAACGGCCAACCTGAGAATAATTACAAGACTACAGTTCCGGT
ACTGGATTCTGATGGCAGTTTTAGGTTGGCGTCCTATTTGACCGTTGATA
AGTCCCGGTGGCAGCAAGGAAATGTTTTTTCCTGTAGTGTGATGCATGAA
GCTCTTCACAATCATTATACGCAAAAGTCTTTGTCCTTGTCCCCTGGGAA
G

SEQ ID NO:9: human IgG1 Fc (human Fc6, mutant)

DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVCTLPPSRDELTKNQVSLSCAVK
GFYPSDIAVEWESNGQPENNYKTTPPVLDSDGSFKLVSKLTVDKSRWQQG
NVFSCSVMHEALHNHYTQKSLSLSPGK

SEQ ID NO:26: human IgG1 Fc (human Fc6, mutant)

GACAAGACCCACACTTGCCCCCCTTGTCCCGCTCCGGAACTCCTGGGCGG
ACCGTCAGTCTTCCTCTTCCCCCCAAAACCCAAGGACACCCTCATGATCT
CCCGGACCCCTGAGGTCACATGCGTGGTGGTGGACGTGAGCCACGAAGAC
CCTGAGGTCAAGTTCAACTGGTACGTGGACGGCGTGGAGGTGCATAATGC
CAAGACAAAGCCGCGGGAGGAGCAGTACAACAGCACGTACCGTGTGGTCA
GCGTCCTCACCGTCCTGCACCAGGACTGGCTGAATGGCAAGGAGTACAAG
TGCAAGGTCTCCAACAAAGCCCTCCCAGCCCCCATCGAGAAAACCATCTC
CAAAGCCAAAGGGCAGCCCCGAGAACCACAGGTGTGTACCCTGCCCCCAT
CCCGGGATGAGCTGACCAAGAACCAGGTCAGCCTGAGTTGCGCGGTCAAA
GGCTTCTATCCCAGCGACATCGCCGTGGAGTGGGAGAGCAATGGGCAGCC
GGAGAACAACTACAAGACCACGCCTCCCGTGTTGGACTCCGACGGCTCCT
TCAAGCTCGTCAGCAAGCTCACCGTGGACAAGAGCAGGTGGCAGCAGGGG
AACGTCTTCTCATGCTCCGTGATGCATGAGGCTCTGCACAACCACTACAC
GCAGAAGAGCCTCTCCCTGTCTCCGGGTAAA

SEQ ID NO:10: human IgG1 Fc (human Fc9, mutant)

DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPCRDELTKNQVSLWCLVK
GFYPSDIAVEWESNGQPENNYKTTPPVLDSDGSFFLYSALTVDKSRWQQG
NVFSCSVMHEALHNHYTQKSLSLSPGK

SEQ ID NO:27: human IgG1 Fc (human Fc9, mutant)

GACAAAACCCACACATGCCCACCTTGTCCCGCCCCTGAACTCCTGGGCGG
ACCGTCAGTCTTCCTCTTCCCCCCAAAACCCAAGGACACCCTCATGATCT
CCCGGACCCCTGAGGTCACATGCGTGGTGGTGGACGTGAGCCACGAAGAC
CCTGAGGTCAAGTTCAACTGGTACGTGGACGGCGTGGAGGTGCATAATGC
CAAGACAAAGCCGCGGGAGGAGCAGTACAACAGCACGTACCGTGTGGTCA
GCGTCCTCACCGTCCTGCACCAGGACTGGCTGAATGGCAAGGAGTACAAG
TGCAAGGTCTCCAACAAAGCCCTCCCAGCCCCCATCGAGAAAACCATCTC
CAAAGCCAAAGGGCAGCCCCGAGAACCACAGGTGTACACCCTGCCCCCAT
GTCGGGATGAGCTGACCAAGAACCAGGTCAGCCTGTGGTGCCTGGTCAAA
GGCTTCTATCCCAGCGACATCGCCGTGGAGTGGGAGAGCAATGGGCAGCC
GGAGAACAACTACAAGACCACGCCTCCCGTGTTGGACTCCGACGGCTCCT
TCTTCCTCTACAGCGCGCTCACCGTGGACAAGAGCAGGTGGCAGCAGGGG
AACGTCTTCTCATGCTCCGTGATGCATGAGGCTCTGCACAACCACTACAC
GCAGAAGAGCCTCTCCCTGTCTCCGGGTAAA

SEQ ID: NO: 11: anti-Claudin antibody heavy chain

QVQLQQPGAELVRPGASVKLSCKASGYTFTSYWINWVKQRPGQGLEWIGN
IYPSDSYTNYNQKFKDKATLTVDKSSSTAYMQLSSPTSEDSAVYYCARVY
YGNSFAYWGQGTTLTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK

SEQ ID: NO: 28: anti-Claudin antibody heavy chain

CAAGTGCAGCTGCAGCAGCCCGGCGCCGAGCTGGTGAGACCCGGCGCCTC
CGTGAAGCTGAGCTGCAAAGCTAGCGGCTACACCTTCACCTCCTACTGGA
TCAACTGGGTGAAGCAGAGACCCGGCCAAGGACTGGAGTGGATCGGCAAC
ATCTACCCCTCCGACTCCTACACCAACTACAACCAGAAGTTTAAGGACAA
GGCCACACTGACCGTGGACAAGTCCTCCAGCACCGCCTACATGCAGCTGT
CCTCCCCTACCTCCGAGGACAGCGCCGTGTACTACTGCGCTAGAGTCTAC
TACGGCAACAGCTTCGCCTACTGGGGCCAAGGCACCACACTGACCGTGAG
CAGCGCCAGCACTAAGGGGCCCTCTGTGTTTCCACTCGCCCCTTCTAGCA
AAAGCACTTCCGGAGGAACTGCCGCTCTGGGCTGTCTGGTGAAAGATTAC
TTCCCCGAACCAGTCACTGTGTCATGGAACTCTGGAGCACTGACATCTGG
AGTTCACACCTTTCCTGCTGTGCTGCAGAGTTCTGGACTGTACTCCCTGT
CATCTGTGGTCACCGTGCCATCTTCATCTCTGGGGACCCAGACCTACATC
TGTAACGTGAACCACAAACCCTCCAACACAAAAGTGGACAAACGAGTCGA
ACCAAAATCTTGTGACAAAACCCACACATGCCCACCTTGTCCCGCCCCTG
AACTCCTGGGCGGACCGTCAGTCTTCCTCTTCCCCCCAAAACCCAAGGAC
ACCCTCATGATCTCCCGGACCCCTGAGGTCACATGCGTGGTGGTGGACGT
GAGCCACGAAGACCCTGAGGTCAAGTTCAACTGGTACGTGGACGGCGTGG
AGGTGCATAATGCCAAGACAAAGCCGCGGGAGGAGCAGTACAACAGCACG
TACCGTGTGGTCAGCGTCCTCACCGTCCTGCACCAGGACTGGCTGAATGG
CAAGGAGTACAAGTGCAAGGTCTCCAACAAAGCCCTCCCAGCCCCCATCG
AGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGAC
CTGCCTGGTCAAAGGCTTCTATCCCAGCGACATCGCCGTGGAGTGGGAGA
GCAATGGGCAGCCGGAGAACAACTACAAGACCACGCCTCCCGTGTTGGAC
TCCGACGGCTCCTTCTTCCTCTACAGCAAGCTCACCGTGGACAAGAGCAG
GTGGCAGCAGGGGAACGTCTTCTCATGCTCCGTGATGCATGAGGCTCTGC
ACAACCACTACACGCAGAAGAGCCTCTCCCTGTCTCCGGGTAAA

SEQ ID: NO: 12: anti-Claudin antibody light chain

DIVMTQSPSSLTVTAGEKVTMSCKSSQSLLNSGNQKNYLTWYQQKPGQPP
KLLIYWASTRESGVPDRFTGSGSGTDFTLTISSVQAEDLAVYYCQNDYFY
PFTFGSGTKLEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC

SEQ ID: NO: 29: anti-Claudin antibody light chain

GACATCGTGATGACCCAGTCCCCTAGCTCTCTGACCGTGACAGCCGGAGA
GAAAGTGACCATGAGCTGCAAGAGCAGCCAATCTCTGCTGAACAGCGGCA
ACCAGAAGAACTATCTGACATGGTATCAGCAGAAGCCCGGCCAACCCCCC
AAGCTGCTGATTTACTGGGCCAGCACAAGAGAGAGCGGAGTGCCCGATAG
GTTCACCGGCAGCGGCAGCGGCACCGATTTCACACTGACCATCTCCAGCG
TGCAAGCCGAAGACCTCGCCGTCTACTACTGCCAGAACGACTACTTCTAC
CCTTTCACCTTCGGCTCCGGCACCAAACTGGAGATCAAGCGTACGGTGGC
TGCACCATCTGTCTTCATCTTCCCGCCATCTGATGAGCAGTTGAAATCTG
GTACCGCTAGCGTTGTGTGCCTGCTGAATAACTTTTATCCACGGGAGGCT
AAGGTGCAGTGGAAAGTGGACAATGCCCTCCAGAGCGGAAATAGCCAAGA
GTCCGTTACCGAACAGGACTCTAAAGACTCTACATACTCCCTGTCCTCCA
CACTGACCCTCTCCAAGGCCGACTATGAGAAACACAAGGTTTACGCATGC
GAGGTCACACACCAGGGACTCTCCTCTCCCGTGACCAAGAGCTTCAACCG
GGGAGAATGC

SEQ ID: NO: 13: anti-Claudin antibody heavy chain-IL-2F42AV1

QVQLQQPGAELVRPGASVKLSCKASGYTFTSYWINWVKQRPGQGLEWIGN
IYPSDSYTNYNQKFKDKATLTVDKSSSTAYMQLSSPTSEDSAVYYCARVY
YGNSFAYWGQGTTLTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGKG
GGGSAPTSSSTKKTQLQLEHLLLDLQMILNGINNYKNPKLTRMLTAKFYM
PKKATELKHLQCLEEELKPLEEVLNLAQSKNFHLEPRDIISNINVIVLEL
KGSETTFMCEYADETATIVEFLNRWITFCQSIISTLT

SEQ ID: NO: 30: anti-Claudin antibody heavy chain-IL-2F42AV1

CAAGTGCAGCTGCAGCAGCCCGGCGCCGAGCTGGTGAGACCCGGCGCCTC
CGTGAAGCTGAGCTGCAAAGCTAGCGGCTACACCTTCACCTCCTACTGGA
TCAACTGGGTGAAGCAGAGACCCGGCCAAGGACTGGAGTGGATCGGCAAC
ATCTACCCCTCCGACTCCTACACCAACTACAACCAGAAGTTTAAGGACAA
GGCCACACTGACCGTGGACAAGTCCTCCAGCACCGCCTACATGCAGCTGT
CCTCCCCTACCTCCGAGGACAGCGCCGTGTACTACTGCGCTAGAGTCTAC
TACGGCAACAGCTTCGCCTACTGGGGCCAAGGCACCACACTGACCGTGAG
CAGCGCCAGCACTAAGGGGCCCTCTGTGTTTCCACTCGCCCCTTCTAGCA
AAAGCACTTCCGGAGGAACTGCCGCTCTGGGCTGTCTGGTGAAAGATTAC
TTCCCCGAACCAGTCACTGTGTCATGGAACTCTGGAGCACTGACATCTGG
AGTTCACACCTTTCCTGCTGTGCTGCAGAGTTCTGGACTGTACTCCCTGT
CATCTGTGGTCACCGTGCCATCTTCATCTCTGGGGACCCAGACCTACATC
TGTAACGTGAACCACAAACCCTCCAACACAAAAGTGGACAAACGAGTCGA
ACCAAAATCTTGTGACAAAACCCACACATGCCCACCTTGTCCCGCCCCTG
AACTCCTGGGCGGACCGTCAGTCTTCCTCTTCCCCCCAAAACCCAAGGAC
ACCCTCATGATCTCCCGGACCCCTGAGGTCACATGCGTGGTGGTGGACGT
GAGCCACGAAGACCCTGAGGTCAAGTTCAACTGGTACGTGGACGGCGTGG
AGGTGCATAATGCCAAGACAAAGCCGCGGGAGGAGCAGTACAACAGCACG
TACCGTGTGGTCAGCGTCCTCACCGTCCTGCACCAGGACTGGCTGAATGG
CAAGGAGTACAAGTGCAAGGTCTCCAACAAAGCCCTCCCAGCCCCCATCG
AGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGAC
CTGCCTGGTCAAAGGCTTCTATCCCAGCGACATCGCCGTGGAGTGGGAGA
GCAATGGGCAGCCGGAGAACAACTACAAGACCACGCCTCCCGTGTTGGAC
TCCGACGGCTCCTTCTTCCTCTACAGCAAGCTCACCGTGGACAAGAGCAG
GTGGCAGCAGGGGAACGTCTTCTCATGCTCCGTGATGCATGAGGCTCTGC
ACAACCACTACACGCAGAAGAGCCTCTCCCTGTCTCCGGGTAAAGGTGGA
GGAGGTAGCGCCCCTACAAGCAGCAGCACCAAGAAGACCCAGCTGCAGCT
GGAACACCTGCTGCTGGATCTGCAGATGATCCTGAACGGCATCAACAACT
ACAAGAACCCCAAGCTGACCCGGATGCTGACCGCCAAGTTCTACATGCCC
AAGAAGGCCACCGAGCTGAAGCACCTCCAGTGTCTGGAGGAGGAGCTGAA
GCCTCTGGAGGAAGTGCTGAACCTGGCCCAGAGCAAGAACTTCCACTTAG
AACCCAGGGACATAATCTCCAACATCAACGTGATAGTGCTGGAACTGAAG
GGCAGCGAGACCACCTTCATGTGCGAGTACGCCGACGAGACCGCTACCAT
CGTGGAGTTCCTGAACCGCTGGATCACCTTTTGCCAGAGCATCATCAGCA
CACTGACC

SEQ ID: NO: 14: anti-Claudin antibody heavy chain-IL-2F42AV2

QVQLQQPGAELVRPGASVKLSCKASGYTFTSYWINWVKQRPGQGLEWIGN
IYPSDSYTNYNQKFKDKATLTVDKSSSTAYMQLSSPTSEDSAVYYCARVY
YGNSFAYWGQGTTLTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGKG
GGSAPTSSSTKKTQLQLEHLLLDLQMILNGINNYKNPKLTRMLTAKFYMP
KKATELKHLQCLEEELKPLEEVLNLAQSKNFHLEPRDTISNINVIVLELK
GSETTFMCEYADETATIVEFLNRWITFCQSIISTLT

SEQ ID: NO: 31: anti-Claudin antibody heavy chain-IL-2F42AV2

CAAGTGCAGCTGCAGCAGCCCGGCGCCGAGCTGGTGAGACCCGGCGCCTC
CGTGAAGCTGAGCTGCAAAGCTAGCGGCTACACCTTCACCTCCTACTGGA
TCAACTGGGTGAAGCAGAGACCCGGCCAAGGACTGGAGTGGATCGGCAAC
ATCTACCCCTCCGACTCCTACACCAACTACAACCAGAAGTTTAAGGACAA
GGCCACACTGACCGTGGACAAGTCCTCCAGCACCGCCTACATGCAGCTGT
CCTCCCCTACCTCCGAGGACAGCGCCGTGTACTACTGCGCTAGAGTCTAC
TACGGCAACAGCTTCGCCTACTGGGGCCAAGGCACCACACTGACCGTGAG
CAGCGCCAGCACTAAGGGGCCCTCTGTGTTTCCACTCGCCCCTTCTAGCA
AAAGCACTTCCGGAGGAACTGCCGCTCTGGGCTGTCTGGTGAAAGATTAC
TTCCCCGAACCAGTCACTGTGTCATGGAACTCTGGAGCACTGACATCTGG
AGTTCACACCTTTCCTGCTGTGCTGCAGAGTTCTGGACTGTACTCCCTGT
CATCTGTGGTCACCGTGCCATCTTCATCTCTGGGGACCCAGACCTACATC
TGTAACGTGAACCACAAACCCTCCAACACAAAAGTGGACAAACGAGTCGA
ACCAAAATCTTGTGACAAAACCCACACATGCCCACCTTGTCCCGCCCCTG
AACTCCTGGGCGGACCGTCAGTCTTCCTCTTCCCCCCAAAACCCAAGGAC
ACCCTCATGATCTCCCGGACCCCTGAGGTCACATGCGTGGTGGTGGACGT
GAGCCACGAAGACCCTGAGGTCAAGTTCAACTGGTACGTGGACGGCGTGG
AGGTGCATAATGCCAAGACAAAGCCGCGGGAGGAGCAGTACAACAGCACG
TACCGTGTGGTCAGCGTCCTCACCGTCCTGCACCAGGACTGGCTGAATGG
CAAGGAGTACAAGTGCAAGGTCTCCAACAAAGCCCTCCCAGCCCCCATCG
AGAAAACCATCTCCAAAGCCAAAGGGCAGCCCCGAGAACCACAGGTGTAC
ACCCTGCCCCCATCCCGGGATGAGCTGACCAAGAACCAGGTCAGCCTGAC
CTGCCTGGTCAAAGGCTTCTATCCCAGCGACATCGCCGTGGAGTGGGAGA
GCAATGGGCAGCCGGAGAACAACTACAAGACCACGCCTCCCGTGTTGGAC
TCCGACGGCTCCTTCTTCCTCTACAGCAAGCTCACCGTGGACAAGAGCAG
GTGGCAGCAGGGGAACGTCTTCTCATGCTCCGTGATGCATGAGGCTCTGC
ACAACCACTACACGCAGAAGAGCCTCTCCCTGTCTCCGGGTAAAGGTGGA
GGAGGTAGCGCCCCTACAAGCAGCAGCACCAAGAAGACCCAGCTGCAGCT
GGAACACCTGCTGCTGGATCTGCAGATGATCCTGAACGGCATCAACAACT
ACAAGAACCCCAAGCTGACCCGGATGCTGACCGCCAAGTTCTACATGCCC
AAGAAGGCCACCGAGCTGAAGCACCTCCAGTGTCTGGAGGAGGAGCTGAA
GCCTCTGGAGGAAGTGCTGAACCTGGCCCAGAGCAAGAACTTCCACTTAG
AACCCAGGGACACAATCTCCAACATCAACGTGATAGTGCTGGAACTGAAG
GGCAGCGAGACCACCTTCATGTGCGAGTACGCCGACGAGACCGCTACCAT
CGTGGAGTTCCTGAACCGCTGGATCACCTTTTGCCAGAGCATCATCAGCA
CACTGACC

SEQ ID NO:15: human IL-2 mutant F42AV2-human IgG1 Fc (human Fc, dimeric, wild-type)

APTSSSTKKTQLQLEHLLLDLQMILNGINNYKNPKLTRMLTAKFYMPKKA
TELKHLQCLEEELKPLEEVLNLAQSKNFHLEPRDTISNINVIVLELKGSE
TTFMCEYADETATIVEFLNRWITFCQSIISTLTGGGSDKTHTCPPCPAPE
LLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALH
NHYTQKSLSLSPGK

SEQ ID NO:32: human IL-2 mutant F42AV2-human IgG1 Fc (human Fc, dimeric, wild-type)

GCCCCTACAAGCAGCAGCACCAAGAAGACCCAGCTGCAGCTGGAACACCT
GCTGCTGGATCTGCAGATGATCCTGAACGGCATCAACAACTACAAGAACC
CCAAGCTGACCCGGATGCTGACCGCCAAGTTCTACATGCCCAAGAAGGCC
ACCGAGCTGAAGCACCTCCAGTGTCTGGAGGAGGAGCTGAAGCCTCTGGA
GGAAGTGCTGAACCTGGCCCAGAGCAAGAACTTCCACTTAGAACCCAGGG
ACACAATCTCCAACATCAACGTGATAGTGCTGGAACTGAAGGGCAGCGAG
ACCACCTTCATGTGCGAGTACGCCGACGAGACCGCTACCATCGTGGAGTT
CCTGAACCGCTGGATCACCTTTTGCCAGAGCATCATCAGCACACTGACCG
GTGGAGGAGGTAGCGACAAGACCCACACTTGCCCCCCTTGTCCCGCTCCG
GAACTCCTGGGCGGACCGTCAGTCTTCCTCTTCCCCCCAAAACCCAAGGA
CACCCTCATGATCTCCCGGACCCCTGAGGTCACATGCGTGGTGGTGGACG
TGAGCCACGAAGACCCTGAGGTCAAGTTCAACTGGTACGTGGACGGCGTG
GAGGTGCATAATGCCAAGACAAAGCCGCGGGAGGAGCAGTACAACAGCAC
GTACCGTGTGGTCAGCGTCCTCACCGTCCTGCACCAGGACTGGCTGAATG
GCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTA
CACCCTGCCCCCATCCCGGGATGAGCTGACCAAGAACCAGGTCAGCCTGA
CCTGCCTGGTCAAAGGCTTCTATCCCAGCGACATCGCCGTGGAGTGGGAG
AGCAATGGGCAGCCGGAGAACAACTACAAGACCACGCCTCCCGTGTTGGA
CTCCGACGGCTCCTTCTTCCTCTACAGCAAGCTCACCGTGGACAAGAGCA
GGTGGCAGCAGGGGAACGTCTTCTCATGCTCCGTGATGCATGAGGCTCTG
CACAACCACTACACGCAGAAGAGCCTCTCCCTGTCTCCGGGTAAA

SEQ ID NO:16: human IgG1 Fc (human Fc, monomeric, wild-type)- human IL-2 mutant F42AV2

APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTKPPSRDELTKNQVSLSCLVKGFYPSDIAVE
WESNGQPENNYKTTVPVLDSDGSFRLASYLTVDKSRWQQGNVFSCSVMHE
ALHNHYTQKSLSLSPGKGGGSAPTSSSTKKTQLQLEHLLLDLQMILNGIN
NYKNPKLTRMLTAKFYMPKKATELKHLQCLEEELKPLEEVLNLAQSKNFH
LEPRDTISNINVIVLELKGSETTFMCEYADETATIVEFLNRWITFCQSII
STLT

SEQ ID NO:33: human IgG1 Fc (human Fc, monomeric, wild-type)- human IL-2 mutant F42AV2

GCGCCGGAGCTGCTCGGTGGACCATCAGTATTCCTCTTCCCACCGAAGCC
TAAGGATACTCTCATGATATCCAGGACTCCTGAGGTTACTTGTGTTGTGG
TTGACGTATCTCATGAGGACCCTGAGGTCAAATTTAATTGGTATGTAGAC
GGAGTGGAAGTCCACAATGCCAAGACTAAACCAAGGGAGGAGCAGTATAA
CTCTACCTACCGCGTTGTGTCAGTGCTGACGGTGTTGCATCAAGACTGGC
TGAACGGGAAGGAATACAAATGTAAGGTGTCCAACAAGGCCCTCCCTGCG
CCTATAGAAAAAACTATAAGTAAAGCCAAAGGTCAACCAAGAGAACCTCA
GGTATATACGAAACCACCCTCCAGGGACGAGCTTACCAAGAACCAGGTTT
CACTGAGTTGTCTCGTCAAGGGGTTCTACCCAAGCGATATTGCGGTCGAG
TGGGAGTCTAATGGACAACCGGAGAATAACTACAAAACTACCGTACCTGT
GCTGGATAGCGATGGGTCTTTCAGACTCGCCTCTTACTTGACGGTCGATA
AAAGTCGATGGCAGCAGGGAAACGTCTTTAGCTGCAGTGTCATGCATGAA
GCACTCCACAACCATTACACTCAGAAAAGTCTTTCTCTGAGTCCAGGTAA
GGGTGGAGGAGGTAGCGCCCCTACAAGCAGCAGCACCAAGAAGACCCAGC TGCAGCTGGAACACCTGCTGCTGGATCTGCAGATGATCCTGAACGGCATC AACAACTACAAGAACCCCAAGCTGACCCGGATGCTGACCGCC AAGTTCTA CATGCCCAAGAAGGCCACCGAGCTGAAGCACCTCCAGTGTCTGGAGGAGG AGCTGAAGCCTCTGGAGGAAGTGCTGAACCTGGCCCAGAGCAAGAACTTC CACTTAGAA CCCAGGGACACA ATCTCCAACATCAACGTGATAGTGCTGGA ACTGAAGGGCAGCGAGACCACCTTCATGTGCGAGTACGCCGACGAGACCG CTACCATCGTGGAGTTCCTGAACCGCTGGATCACCTTTTGCCAGAGCATC ATCAGCACACTGACC

SEQ ID NO:17: human IL-2 mutant F42AV2-human IgG1 Fc (human Fc, dimeric, wild-type)- anti-Claudin antibody heavy chain

APTSSSTKKTQLQLEHLLLDLQMILNGINNYKNPKLTRMLTAKFYMPKKA
TELKHLQCLEEELKPLEEVLNLAQSKNFHLEPRDTISNINVIVLELKGSE
TTFMCEYADETATIVEFLNRWITFCQSIISTLTGGGSDKTHTCPPCPAPE
LLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALH
NHYTQKSLSLSPGKGGGSQVQLQQPGAELVRPGASVKLSCKASGYTFTSY
WINWVKQRPGQGLEWIGNIYPSDSYTNYNQKFKDKATLTVDKSSSTAYMQ
LSSPTSEDSAVYYCARVYYGNSFAYWGQGTTLTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKRVEPKSC

SEQ ID NO:34: human IL-2 mutant F42AV2-human IgG1 Fc (human Fc, dimeric, wild-type)- anti-Claudin antibody heavy chain (mutations in bold)

GCCCCTACAAGCAGCAGCACCAAGAAGACCCAGCTGCAGCTGGAACACCT
GCTGCTGGATCTGCAGATGATCCTGAACGGCATCAACAACTACAAGAACC
CCAAGCTGACCCGGATGCTGACCGCCAAGTTCTACATGCCCAAGAAGGCC
ACCGAGCTGAAGCACCTCCAGTGTCTGGAGGAGGAGCTGAAGCCTCTGGA
GGAAGTGCTGAACCTGGCCCAGAGCAAGAACTTCCACTTAGAACCCAGGG
ACACAATCTCCAACATCAACGTGATAGTGCTGGAACTGAAGGGCAGCGAG
ACCACCTTCATGTGCGAGTACGCCGACGAGACCGCTACCATCGTGGAGTT
CCTGAACCGCTGGATCACCTTTTGCCAGAGCATCATCAGCACACTGACCG
GTGGAGGAGGTAGCGACAAGACCCACACTTGCCCCCCTTGTCCCGCTCCG
GAACTCCTGGGCGGACCGTCAGTCTTCCTCTTCCCCCCAAAACCCAAGGA
CACCCTCATGATCTCCCGGACCCCTGAGGTCACATGCGTGGTGGTGGACG
TGAGCCACGAAGACCCTGAGGTCAAGTTCAACTGGTACGTGGACGGCGTG
GAGGTGCATAATGCCAAGACAAAGCCGCGGGAGGAGCAGTACAACAGCAC
GTACCGTGTGGTCAGCGTCCTCACCGTCCTGCACCAGGACTGGCTGAATG
GCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTA
CACCCTGCCCCCATCCCGGGATGAGCTGACCAAGAACCAGGTCAGCCTGA
CCTGCCTGGTCAAAGGCTTCTATCCCAGCGACATCGCCGTGGAGTGGGAG
AGCAATGGGCAGCCGGAGAACAACTACAAGACCACGCCTCCCGTGTTGGA
CTCCGACGGCTCCTTCTTCCTCTACAGCAAGCTCACCGTGGACAAGAGCA
GGTGGCAGCAGGGGAACGTCTTCTCATGCTCCGTGATGCATGAGGCTCTG
CACAACCACTACACGCAGAAGAGCCTCTCCCTGTCTCCGGGTAAAGGTGG
AGGAGGTAGCCAAGTGCAGCTGCAGCAGCCCGGCGCCGAGCTGGTGAGAC
CCGGCGCCTCCGTGAAGCTGAGCTGCAAAGCTAGCGGCTACACCTTCACC
TCCTACTGGATCAACTGGGTGAAGCAGAGACCCGGCCAAGGACTGGAGTG
GATCGGCAACATCTACCCCTCCGACTCCTACACCAACTACAACCAGAAGT
TTAAGGACAAGGCCACACTGACCGTGGACAAGTCCTCCAGCACCGCCTAC
ATGCAGCTGTCCTCCCCTACCTCCGAGGACAGCGCCGTGTACTACTGCGC
TAGAGTCTACTACGGCAACAGCTTCGCCTACTGGGGCCAAGGCACCACAC
TGACCGTGAGCAGCGCCAGCACTAAGGGGCCCTCTGTGTTTCCACTCGCC
CCTTCTAGCAAAAGCACTTCCGGAGGAACTGCCGCTCTGGGCTGTCTGGT
GAAAGATTACTTCCCCGAACCAGTCACTGTGTCATGGAACTCTGGAGCAC
TGACATCTGGAGTTCACACCTTTCCTGCTGTGCTGCAGAGTTCTGGACTG
TACTCCCTGTCATCTGTGGTCACCGTGCCATCTTCATCTCTGGGGACCCA
GACCTACATCTGTAACGTGAACCACAAACCCTCCAACACAAAAGTGGACA
AACGAGTCGAACCAAAATCTTGT

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. An IL-2 receptor binding fusion protein comprising:
an IL-2 mutant protein that binds an IL-2 Receptor; and
an antibody Fc fragment;
wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4.

2. The fusion protein of claim 1, further comprising a tumor-targeting antibody or a tumor-specific protein binding domain at a carboxy- or amino-terminus.

3. The fusion protein of claim 1, wherein the IL-2 mutant has a higher affinity for the IL-2 Receptor at pH 6.9 than IL-2.

4. The fusion protein of claim 1, wherein the IL-2 mutant has a higher affinity for the IL-2 Receptor at pH 6.4.

5. The fusion protein of claim 1, wherein the IL-2 mutant has an F42A mutation that eliminated binding to IL-2 Receptor alpha.

6. The fusion protein of claim 2, wherein a tumor antigen targeted is selected from HER1, HER2, HER3, GD2, carcinoembryonic antigens (CEAs), epidermal growth factor receptor active mutant (EGFRVIII), CD133, Fibroblast Activation Protein Alpha (FAP), Epithelial cell adhesion molecular (Epcam), Glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), or a Claudin family protein.

7. The fusion protein of claim 2, wherein a tumor is selected from the group consisting of: acute myeloid leukemia, adrenocortical carcinoma, B-cell lymphoma, bladder urothelial carcinoma, breast ductal carcinoma, breast lobular carcinoma, esophageal carcinomas, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin’s lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma.

8. The fusion protein of claim 1, wherein the fusion protein comprises human sequences;
comprises, in order from amino to carboxy, the IL-2 mutant protein and the antibody Fc fragment;
comprises, in order from amino to carboxy, the antibody Fc fragment and the IL-2 mutant protein;
further comprising one or more protein domains at an amino-, a carboxy-, or both the amino- and carboxy-terminus of the fusion protein, wherein the one or more protein domains is an antibody, binding fragments thereof, Fc region, cytokine or receptor;
wherein the Fc is a wild-type or a mutated Fc; or
the fusion protein has at least 90, 91, 92, 93, 94, 95, 96, 97, 98, 99, or 100% sequence identity with SEQ ID NO: 13, 14, 15, 16, or 17.

9. A method of treating a cancer patient providing a subject in need thereof an IL-2 receptor binding fusion protein comprising:
providing the cancer patient with an effective amount of the IL-2 receptor binding fusion protein comprising:
an IL-2 mutant protein that binds the IL-2 receptor; and
an antibody Fc fragment;
wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4.

10. The method of claim 9, further comprising a tumor-targeting antibody or a tumor-specific protein binding domain at the carboxy- or amino terminus.

11. The method of claim 9, wherein the IL-2 mutant has a higher affinity for the IL-2 Receptor at pH 6.9 than IL-2.

12. The method of claim 9, wherein the IL-2 mutant has a higher affinity for the IL-2 Receptor at pH 6.4.

13. The method of claim 9, wherein the IL-2 mutant has an F42A mutation that eliminated binding to IL-2 Receptor alpha.

14. The method of claim 10, wherein a tumor antigen targeted is selected from HER1, HER2, HER3, GD2, carcinoembryonic antigens (CEAs), epidermal growth factor receptor active mutant (EGFRVIII), CD133, Fibroblast Activation Protein Alpha (FAP), Epithelial cell adhesion molecular (Epcam), Glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), or a Claudin family protein.

15. The method of claim 10, wherein a tumor is selected from the group consisting of: acute myeloid leukemia, adrenocortical carcinoma, B-cell lymphoma, bladder urothelial carcinoma, breast ductal carcinoma, breast lobular carcinoma, esophageal carcinomas, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin’s lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma.

16. The method of claim 10, wherein the fusion protein at least one of:
comprises human sequences;
comprises, in order from amino to carboxy, the IL-2 mutant protein and the antibody Fc fragment;
comprises, in order from amino to carboxy, the antibody Fc fragment and the IL-2 mutant protein;
further comprising one or more protein domains at an amino-, a carboxy-, or both the amino- and carboxy-terminus of the fusion protein, wherein the one or more protein domains is an antibody, binding fragments thereof, Fc region, cytokine or receptor;
wherein the Fc is a wild-type or a mutated Fc; or
the fusion protein has at least 90, 91, 92, 93, 94, 95, 96, 97, 98, 99, or 100% sequence identity with SEQ ID NO: 13, 14, 15, 16, or 17.

17. The method of claim 10, wherein the fusion protein has SEQ ID NOS: 3 or 4, or at least 90, 91, 92, 93, 94, 95, 96, 97, 98, 99, or 100% sequence identity with SEQ ID NO: 13, 14, 15, 16, or 17.

18. A nucleic acid that encodes an IL-2 receptor binding fusion protein comprising:
an IL-2 mutant protein that binds the IL-2 receptor; and
an antibody Fc fragment;
wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4.

19. The nucleic acid of claim 18, further comprising a tumor-targeting antibody or a tumor-specific protein binding domain at a carboxy- or amino-terminus.

20. The nucleic acid of claim 1818, wherein the IL-2 mutant has a higher affinity for the IL-2 Receptor at pH 6.9 than IL-2.

21. The nucleic acid of claim 18, wherein the IL-2 mutant has a higher affinity for the IL-2 Receptor at pH 6.4.

22. The nucleic acid of claim 18, wherein the IL-2 mutant has an F42A mutation that eliminated binding to IL-2 Receptor alpha.

23. The nucleic acid of claim 19, wherein a tumor antigen targeted is selected from HER1, HER2, HER3, GD2, carcinoembryonic antigens (CEAs), epidermal growth factor receptor active mutant (EGFRVIII), CD133, Fibroblast Activation Protein Alpha (FAP), Epithelial cell adhesion molecular (Epcam), Glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), or a Claudin family protein.

24. The nucleic acid of claim 18, wherein a tumor is selected from the group consisting of: acute myeloid leukemia, adrenocortical carcinoma, B-cell lymphoma, bladder urothelial carcinoma, breast ductal carcinoma, breast lobular carcinoma, esophageal carcinomas, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin’s lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma.

25. The nucleic acid of claim 18, wherein the fusion protein comprises at least one of:
human sequences;
comprises, in order from amino to carboxy, the IL-2 mutant protein and the antibody Fc fragment;
comprises, in order from amino to carboxy, the antibody Fc fragment and the IL-2 mutant protein;
further comprising one or more protein domains at an amino-, a carboxy-, or both the amino- and carboxy-terminus of the fusion protein, wherein the one or more protein domains is an antibody, binding fragments thereof, Fc region, cytokine or receptor;
wherein the Fc is a wild-type or a mutated Fc;
the fusion protein with has at least 90, 95, 96, 97, 98, 99, or 100% sequence identity to SEQ ID NOS: 3 or 4; or
the fusion protein has at least 90, 91, 92, 93, 94, 95, 96, 97, 98, 99, or 100% sequence identity with SEQ ID NO: 13, 14, 15, 16, or 17.

26. A vector that expresses a nucleic acid that encodes an IL-2 receptor binding fusion protein comprising:
an IL-2 mutant protein that binds the IL-2 receptor; and
an antibody Fc fragment;
wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4.

27. A host cell that comprises a vector that expresses a nucleic acid that encodes an IL-2 receptor binding fusion protein comprising:
an IL-2 mutant protein that binds the IL-2 receptor; and
an antibody Fc fragment;
wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4.

28. A method of making an IL-2 receptor binding fusion protein comprising:
expressing a nucleic acid that encodes the IL-2 receptor binding fusion protein comprising:
an IL-2 mutant protein that binds the IL-2 receptor; and
an antibody Fc fragment;
wherein IL-2 mutant protein binds and maintains its activity through the IL-2 receptor at a low pH and wherein the fusion protein has at least 90, 95, 96, 97, 98, 99, or 100% sequence identity to SEQ ID NOS: 3 or 4.
